(19) United States
(12) Patent Application Publication
(10) Pub. No.: US 2011/0251099 A1
(43) Pub. Date: Oct. 13, 2011

(54) SERUM MARKERS PREDICTING CLINICAL RESPONSE TO ANTI-TNFa ANTIBODIES IN PATIENTS WITH ANKYLOSING SPONDYLITIS

(76) Inventors: Sudha Visvanathan, Hoboken, NJ (US); Carrie Wagner, Chesterbrook, PA (US)

(21) Appl. No.: 13/140,441

(22) PCT Filed: Dec. 9, 2009
(86) PCT No.: PCT/US09/67282

§ 371 Date: Jun. 17, 2011

Related U.S. Application Data
(60) Provisional application No. 61/141,421, filed on Dec. 30, 2008.

(51) Int. Cl. C40B 30/04; G06F 19/10; C40B 60/08; G01N 30/00; C12Q 1/42; C12M 1/34

(52) U.S. Cl. 506/9; 435/21; 435/287.2; 506/37; 422/69; 702/19

(57) ABSTRACT

The invention provides tools for management of patients diagnosed with ankylosing spondylitis and prior to the initiation of therapy with an anti-TNFalpha agent. The tools are specific markers and algorithms of predicting response to therapy based on standard clinical primary and secondary end-points using serum marker concentrations. In one embodiment the baseline level of leptin or osteocalcin is used to predict the response at Week 14 after the initiation of therapy. In another embodiment, the change in a serum protein biomarker after 4 weeks of therapy is used such as complement component 3.

### PRIOR APPLICATION

This application claims priority to U.S. application Ser. No. 61/141,421, filed Dec. 30, 2008, which is entirely incorporated herein by reference.

### BACKGROUND OF THE INVENTION

1. Field of the Invention

The present invention relates to methods and procedures for the use of serum biomarkers to predict the response of patients diagnosed with ankylosing spondylitis to treatment with anti-TNFalpha biologic therapeutics.

2. Description of the Related Art

The decision to treat ankylosing spondylitis (AS) with biologics currently available or which are in development such as golimumab or adalimumab, human anti-TNFalpha antibodies, or infliximab, a murine-human chimeric anti-TNFa antibody, or enteracept, a TNFR construct, presents a number of challenges. One of the challenges is predicting which subjects will respond to treatment and which subjects will lose response following treatment.

Biomarkers are defined as “a characteristic that is objectively measured and evaluated as an indicator of normal biologic processes, pathogenic processes, or pharmacologic responses to a therapeutic intervention” Biomarker Working Group, 2001. Clin. Pharm. and Therap. 69: 89-95). The definition of a biomarker has recently been further defined as proteins the change of expression of which may correlate with an increased risk of disease or progression, or which may be predictive of a response to a given treatment.

Neutralization of TNFalpha through the addition of an anti-TNFa antibody to in vitro or in vivo systems, can modify the expression of inflammatory cytokines and a number of other serum protein and non-protein components. An anti-TNFa antibody added to cultured synovial fibroblasts reduced the expression of the cytokines IL-1, IL-6, IL-8, and GM-CSF (Feldmann & Maini (2001) Annu Rev Immunol 19:163-196). RA patients who were treated with infliximab had decreased serum levels of TNFR1, TNFR2, IL-1R antagonist, IL-6, serum amyloid A, haptoglobin, and fibrinogen (Charles 1999 J Immunol 163:1521-1528). Other studies have shown that RA patients who are treated with infliximab had decreased serum levels of soluble(s) ICAM-3 and sP-selectin (Gonzalez-Gay, 2006 Clin Exp Rheumatol 24: 373-379), as well as a reduction in the levels of the cytokine IL-18 (Pittoni, 2002 Ann Rheum Dis 61:723-725; van Oosterhout, 2005 Ann Rheum Dis 64:537-543).

Elevated levels of C-reactive protein (CRP) have been observed in patients with various immune-mediated inflammatory diseases. These observations indicate that CRP may have potential value as a marker anti-TNFa treatment. (St Clair, 2004 Arthritis Rheum 50:3432-3443) showed that infliximab returned CRP to normal levels in patients with early RA. In refractory psoriatic arthritis (Feletar, 2004 Ann Rheum Dis 63:156-161), treatment with infliximab also returned CRP to normal levels. CRP levels have also been shown to be associated with joint damage progression in early RA patients treated only with methotrexate (Smolen, 2006 Arthritis Rheum 54:702-710). When infliximab treatment was added to the methotrexate treatment, the CRP levels were no longer associated with the progression of joint damage.

In the treatment of patients with RA, Charles (1999) and Strunk (2006 Rheumatol Int. 26: 252-256) demonstrated that infliximab could reduce the expression of inflammation-related cytokines such as IL-6, as well as angiogenesis related cytokines such as VEGF (vascular endothelial growth factor). Ulfgren (2000 Arthritis Rheum 43:2391-2396) showed that infliximab treatment reduced the synthesis of TNF, IL-1α, and IL-1beta in the synovium within 2 weeks of treatment. Mastroianni (2005 Br J Dermatol 153:531-536) showed that reductions in VEGF, FGF, and MMP-2 were significant improvement in the area and severity of psoriasis following treatment with infliximab. Visvanathan (Ann Rheum Dis 2008; 67;511-517) showed that infliximab treatment reduced the levels of IL-6, VEGF, and CRP in the serum of AS patients, and that the reductions reflected improved disease activity measures.

Treatment of AS patients with infliximab caused decreases in IL-6 that were associated with improved clinical measures (Visvanathan, 2006 Arthritis Rheum 54(Suppl): S792). In the infliximab treated patients, early decreases in IL-6 and CRP following treatment were associated with improvement in disease activity scores.

Pre-treatment serum marker concentrations have also been associated with response to anti-TNFa treatment. A low baseline serum level of IL-2R was found to be associated with clinical response to infliximab in patients with refractory RA (Kuuliala 2006). Visvanathan (2007a) showed that the treatment of RA patients with infliximab plus MTX induced a decrease in a number of inflammation-related markers, including MMP-3. It was shown in this study that at baseline the levels of MMP-3 correlated significantly with measures of clinical improvement one year post-treatment.

Therefore, while a number of serum protein and non-protein markers of inflammation and systemic disease have been demonstrated to be modified during anti-TNFa treatment, a unique set of markers and a predictive algorithm has not, thus far, been discovered.

### SUMMARY OF THE INVENTION

The invention relates the use of multiple biomarkers to predict the response of a patient to treatment with anti-TNFα, and more specifically, to determine if a patient will or will not respond. In addition, the invention can be used to determine if a patient has responded to treatment, and if the response will be sustained. In one aspect, the invention encompasses the use of a multi-component screen using patient serum samples, to predict the response as well as non-response of patients with AS to treatment with a TNFα neutralizing monoclonal antibody.

In one embodiment, specific marker sets identified in datasets from patients with AS prior to the initiation of anti-TNFalpha therapy, having been correlated to actual clinical response assessment, are used to predict clinical response of AS patients prior to treatment with anti-TNFalpha therapy. In a specific embodiment the marker set is two or more markers chosen from the group consisting of leptin, TIMP-1, CD40 ligand, G-CSF, MCP-1, osteocalcin, PAP, complement component 3, VEGF, insulin, ferritin, and ICAM-1.

In another embodiment, specific marker sets identified in datasets from patients with AS prior to and following the initiation of anti-TNFalpha therapy, having been correlated to actual clinical response assessment, are used to predict clinical response of AS patients prior to treatment with anti-TNFalpha therapy. In a specific embodiment the marker set is two or more markers chosen from the group consisting of leptin, TIMP-1, CD40 ligand, G-CSF, MCP-1, osteocalcin, PAP, complement component 3, VEGF, insulin, ferritin, and ICAM-1.

The invention also provides a computer-based system for predicting the response of an AS patient to anti-TNFalpha therapy wherein the computer uses values from a patient's dataset to compare to a predictive algorithm, such as a decision tree, wherein the dataset includes the serum concentrations of one or more markers selected from the group consisting of leptin, TIMP-1, CD40 ligand, G-CSF, MCP-1, osteocalcin, PAP, complement component 3, VEGF, insulin, ferritin, and ICAM-1. In one embodiment, the computer-based system is a trained neural network for processing a patient dataset and producing an output wherein the dataset includes one or more serum marker concentrations selected from the group consisting of leptin, TIMP-1, CD40 ligand, G-CSF, MCP-1, osteocalcin, PAP, insulin, complement component 3, VEGF, and ICAM-1 .

The invention also provides a device capable of processing and detecting serum markers in a specimen or sample obtained from an AS patient wherein the serum marker concentrations selected from the group consisting of leptin, TIMP-1, CD40 ligand, G-CSF, MCP-1, osteocalcin, PAP, complement component 3, VEGF, insulin, ferritin, and ICAM-1.

The invention also provides a kit comprising a device capable of processing and detecting serum markers in a specimen or sample obtained from an AS patient wherein the serum marker concentrations selected from the group consisting of leptin, TIMP-1, CD40 ligand, G-CSF, MCP-1, osteocalcin, PAP, complement component 3, VEGF, insulin, ferritin, and ICAM-1.

### BRIEF DESCRIPTION OF THE SEVERAL VIEWS OF THE DRAWING

FIGS. 1-6 are AS response prediction models shown in the form of a decision tree based on the use of serum biomarkers and correlated to patient clinical responses assessed by ASAS20 or BASDAI. The non-responder or “No” node means all subjects in that node are predicted by the model to be non-responders, while a “Yes” node means all subjects in that node are predicted by the model to be responders. Within the node, the number of actual non-responders/number of actual responders in that node is shown.

FIG. 1 is a predictive model developed from baseline (Week 0) marker data analyzed by multiplexed method from study patients receiving golimumab using the ASAS20 at Week 14, where initial classifier for a responder is based on leptin (cutoff value<3.804, log scale) and the secondary classifier for a responder is based on CD40 ligand (a cutoff value>=1.05, log scale).

FIG. 2 is a predictive model developed from baseline (Week 0) marker data analyzed by multiplexed method from study patients receiving golimumab using the change in BASDAI at Week 14 where the initial responder criteria is TIMP-1 (cutoff value>=7.033) and the secondary classifier of a responder is G-CSF (cutoff value<3.953); when TIMP-1 is below the cutoff value, prostatic acid phosphatase is a classifier for responders (cutoff>=−1.287, log value); when TIMP-1 and PAP are both below their respective cutoff values, MCP-1 is a classifier for responders (<7.417, log scale).

FIG. 3 is a AS response prediction model developed from serum marker values at baseline (Week 0) quantitated by both multiplex methods and individual EIA from study patients receiving golimumab and responses assessed using ASAS20 at Week 14, where the osteocalcin is the initial classifier of a responder (cutoff value>=3.878,log scale), and when osteocalcin is below its respective cutoff value, PAP is used as a classifier of a responder (cutoff value>=−1.359, log scale).

FIG. 4 is a AS response prediction model developed from serum marker values at baseline (Week 0) quantitated by both multiplex methods and individual EIA from study patients receiving golimumab and responses assessed using BASDAI change at Week 14, where osteocalcin is the initial classifier of a responder (cutoff value>=3.977, log scale), and when osteocalcin is below the cutoff value, PAP is a classifier of a responder (cutoff>=−1.415), and when both osteocalcin and PAP are below their respective cutoff values, insulin is used as a classifier of a responder (cutoff value<2.711, log scale).

FIG. 5 is a AS response prediction model developed from baseline and the change in serum marker values from baseline (Week 0) to Week 4 after initiation of anti-TNF therapy quantitated by multiplex methods from study patients receiving golimumab and responses were assessed using ASAS20 at Week 14, where baseline leptin is the initial classifier of a responder (cutoff value<3.804, log scale), and when leptin is below its cutoff value, the change if complement 3 from baseline to Week 4 is used as a classifier of a responder (cutoff<−0.224), and when both leptin and complement 3 are equal to or above their respective cut off values, baseline VEGF is used as a classifier of a responder (cutoff>=8.724).

FIG. 6 is a AS response prediction model developed from the baseline and the change in serum marker values from baseline (Week 0) to Week 4 after initiation of anti-TNF therapy quantitated by multiplex methods from study patients receiving golimumab and responses assessed using change in BASDAI at Week 14, where the initial responder criteria is the change complement component 3 from baseline to Week 4 (cutoff value<−0.233, log scale), and when change in complement 3 is equal to or above the cutoff value, baseline ferritin is used as a classifier (cutoff value>=7.774, log scale), and when change in complement 3 is equal to or above the cutoff value and baseline ferritin is below its respective cutoff value, the change in ICAM-1 is used as a classifier of a responder (cutoff value>=−0.2204, log scale).

### DETAILED DESCRIPTION OF THE INVENTION

### Abbreviations

ASAS: Ankylosing Spondylitis Assessment
BASDAI: Bath Ankylosing Spondylitis Disease Activity Index
BASMI: Bath Ankylosing Spondylitis Metrology Index
BASFI: Bath Ankylosing Spondylitis Functional Index
CART classification and regression tree model
EIA Enzyme Immunoassay
ELISA Enzyme Linked Immunoassay G-CSF granulocyte colony stimulating factor
MAP multi-analyte profile
PAP prostatic acid phosphatase
SELDI Surface Enhanced Laser Desorption and Ionization
SA serum amyloid P component this is not a common abbreviation for serum amyloid P
TNFα/TNFa Tumor Necrosis Factor alpha
TNFR Tumor Necrosis Factor receptor
IL Interleukin
IL-1R IL-1 receptor

### Definitions

A “biomarker” is defined as ‘[a] characteristic that is objectively measured and evaluated as an objective indicator of normal biological processes, pathogenic processes, or pharmacologic responses to a therapeutic intervention’ by the Biomarkers Definitions Working Group (Atkinson et al. 2001 Clin Pharm Therap 69(3):89-95). Thus, an anatomic or physiologic process can serve as a biomarker, for example, range of motion, as can levels of proteins, gene expression (mRNA), small molecules, metabolites or minerals, provided there is a validated link between the biomarker and a relevant physiologic, toxicologic, pharmacologic, or clinical outcome.

By “serum level” of a marker is meant the concentration of the marker measured by one or more methods, such as an immunoassay, typically ex vivo on a sample prepared from a specimen such as blood. The immunoassay uses immunospecific reagents, typically antibodies, for each marker and the assay may be performed in a variety of formats including enzyme-coupled reactions, e.g. EIA, ELISA, RIA, or other direct or indirect probe. Other methods of quantitating the marker in the sample such electrochemical, fluorescence probe-linked detection are also possible. The assay may also be “multiplexed” wherein multiple markers are detected and quantitated during a single sample interrogation.

Observational studies usually report their results as odds ratios (OR) or relative risks. Both are measures of the size of an association between an exposure (e.g., smoking, use of a medication) and a disease or death. A relative risk of 1.0 indicates that the exposure does not change the risk of disease. A relative risk of 1.75 indicates that patients with the exposure are 1.75 times more likely to develop the disease or have a 75 percent higher risk of disease. A relative risk of less than 1 indicates that the exposure decreases risk. Odds ratios are a way to estimate relative risks in case-control studies, when the relative risks cannot be calculated specifically. Although it is accurate when the disease is rare, the approximation is not as good when the disease is common.

Predictive values help interpret the results of tests in the clinical setting. The diagnostic value of a procedure is defined by its sensitivity, specificity, predictive value and efficiency. Any test method will produce True Positive (TP), False Negative (FN), False Positive (FP), and True Negative (TN). “Sensitivity” of a test is the percentage of all patients with disease present or that do respond who have a positive test or (TP/TP+FN)×100%. “Specificity” of a test is the percentage of all patients without disease or who do not respond, who have a negative test or (TN/FP+TN)×100%. The “predictive value” or “PV” of a test is a measure (%) of the times that the value (positive or negative) is the true value, i.e. the percent of all positive tests that are true positives is the Positive Predictive Value (PV+) or (TP/TP+FP)×100%. The “negative predictive value” (PV−) is the percentage of patients with a negative test who will not respond or (TN/FN+TN)×100%. The “accuracy” or “efficiency” of a test is the percentage of the times that the test give the correct answer compared to the total number of tests or (TP+TN/TP+TN+FP+FN)×100%. The “error rate” is when patients predicted to respond do not and patients not predicted to respond or (FP+FN/TP+TN+FP+FN)×100%. The overall test “specificity” is a measure of the accuracy of the sensitivity and specificity of a test do not change as the overall likelihood of disease changes in a population, the predictive value does change. The PV changes with a physician's clinical assessment of the presence or absence of disease or presence or absence of clinical response in a given patient.

A “decreased level” or “lower level” of a biomarker refers to a level that is quantifiably less relative to a predetermined value called the “cutoff value “and above the limit of quantitation (LOQ)”, which “cutoff value” is specific for the algorithm and parameters related to patient sampling and treatment conditions.

A “higher level” or “elevated level” of a biomarker refers to a level that is quantifiably elevated relative to a predetermined value called the “cutoff value”, which “cutoff value” is specific for the algorithm and parameters related to patient sampling and treatment conditions.

The term “human TNFα” (abbreviated herein as hTNFalpha, hTNFa or simply TNF), as used herein, is intended to refer to a human cytokine that exists as a 17 kD secreted form and a 26 kD membrane associated form, the biologically active form of which is composed of a trimer of noncovalently bound 17 kD molecules. The term human TNFα is intended to include recombinant human TNFα (rhTNFα), which can be prepared by standard recombinant expression methods or purchased commercially (R & D Systems, Catalog No. 210-TA, Minneapolis, Minn.).

By “anti-TNFa”, “anti-TNFα”, anti-TNFalpha or simply “anti-TNF” therapy or treatment is meant the administration to a patient of a biologic molecule (biopharmaceutical) capable of blocking, inhibiting, neutralizing, preventing receptor binding, or preventing TNFR activation by TNFα. Examples of such biopharmaceuticals are neutralizing Mabs to TNFα including but not limited those antibodies sold under the generic names of infliximab and adalimumab, and antibodies in clinical development such as golimumab; also included are non-antibody constructs capable of binding TNFa such as the TNFR-immunoglobulin chimera known as enteracept. The term includes each of the anti-TNFα human antibodies and antibody portions described herein as well as those described in U.S. Pat. Nos. 6,090,382; 6,258,562; 6,509,015, and in U.S. patent application Ser. Nos. 09/801,185 and 10/302,356. In one embodiment, the TNFα inhibitor used in the invention is an anti-TNFα antibody, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; and Centocor, see WO 02/12502), and adalimumab (Humira® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein. In another embodiment, the TNFα inhibitor is a TNF fusion protein, e.g., etanercept (Enbrel®, Amgen; described in WO 91/03553 and WO 09/406476, incorporated by reference herein). In another embodiment, the TNFα inhibitor is a recombinant TNF binding protein (r-TBP-I) (Serono).

By “sample” or “patient's sample” is meant a specimen which is a cell, tissue, or fluid or portion thereof extracted, produced, collected, or otherwise obtained from a patient suspected to having or having presented with symptoms associated with a TNFalpha-related disease.

### Overview

Recent advances in technologies such as proteomics present pathologists with the challenge of integrating the new information generated with high-throughput methods with current diagnostic models based on clinicopathologic correlations and often with the inclusion of histopathological findings. Parallel developments in the field of medical informatics and bioinformatics provide the technical and mathematical methods to approach these problems in a rational manner providing new tools to the practitioner and pathologist or other medical specialists in the form multivariate and multidisciplinary diagnostic and prognostic models that are hoped to provide more accurate, individualized patient-based information. Evidence-based medicine (EBM) and medical decision analysis (MDA) are among these relatively new disciplines that use quantitative methods to assess the value of information and integrate so-called best evidence into multivariate models for the assessment of prognosis, response to therapy, and selection of laboratory tests that can influence individual patient care.

This invention includes several aspects:
1. The use of serum to identify biomarkers associated with the response or non-response to anti-TNF, such as golimumab, treatment in patients with AS.
2. The ability to predict a response or non-response to an anti-TNFalpha Mab, such as golimumab, treatment using biomarkers present in serum from a diagnosed AS patient prior to initiating anti-TNF therapy.
3. An algorithm to predict outcome in patients with AS treated with anti-TNF therapy
   a. The clinical response or non-response of AS patients to anti-TNFα at Week 14 may be predicted at the time of assessment (Week 0) using biomarkers present in a diagnosed AS patient's serum prior to the initiation of anti-TNF therapy.
   b. The clinical response or non-response of AS patients to anti-TNFa treatment at Week 14 may be predicted using the change in biomarkers from a baseline value obtained prior to the initiation of therapy (Week 0) and at Week 4 after initiation of therapy.
   c. The clinical response or non-response of AS patients to anti-TNFa treatment at Week 14 may be predicted using the change in biomarkers from a baseline value obtained prior to the initiation of therapy (Week 0) in combination with the change in biomarkers at Week 4 after initiation of therapy.
4. Devices, systems, and kits comprising means for using the markers of the invention to predict response or non-response of an AS patient to anti-TNFa therapy.

In order to define the markers useful in developing a predictive algorithm based on the concentrations of markers, serum was obtained from patients who had been treated with golimumab. Serum can be obtained at baseline (Week 0), Week 4 and Week 14 of treatment or other intermediate or longer time points. A number of biomarkers in the serum samples are analyzed, and the baseline concentration as well as the change in the concentration of biomarkers after treatment is determined The baseline and change in biomarker expression is then used to determine if the biomarker expression correlates with the treatment outcome at Week 14 or other defined timepoint after the initiation of treatment as assessed by the ASAS20 or another measure of clinical response. In one embodiment, the process for defining the markers associated with the clinical response of a patient with AS to anti-TNFalpha therapy and developing an algorithm for predicting response or non-response involving the serum concentrations of those markers uses a stepwise analysis wherein the initial correlations are done by logistic regression analysis relating the value for each biomarker for each patient at Week 0, 4, and 14 to the clinical assessment for that patient at Week 14 and 24 and once the ability of a marker to significantly correlate to response to therapy at multiple clinical endpoints is determined, a unique algorithm based on defined serum values of a marker or marker set is developed using CART or other suitable analytic method as described herein or known in the art.

In addition to the other markers disclosed herein, the dataset markers may be selected from one or more clinical indicia, examples of which are age, gender, blood pressure, height and weight, body mass index, CRP concentration, tobacco use, heart rate, fasting insulin concentration, fasting glucose concentration, diabetes status, use of other medications, and specific functional or behavioral assessments, and/or radiological or other image-based assessments wherein a numerical values are applied to individual measures or an overall numerical score is generated. Clinical variables will typically be assessed and the resulting data combined in an algorithm with the above described markers.

Prior to input into the analytical process, the data in each dataset is collected by measuring the values for each marker, usually in triplicate or in multiple triplicates. The data may be manipulated, for example, raw data may be transformed using standard curves, and the average of triplicate measurements used to calculate the average and standard deviation for each patient. These values may be transformed before being used in the models, e.g. log-transformed, Box-Cox transformed (see Box and Cox (1964) J. Royal Stat. Soc, Series B, 26:211 & #8212;246), etc. This data can then be input into the analytical process with defined parameters.

The quantitative data thus obtained related to the protein markers and other dataset components is then subjected to an analytic process with parameters previously determined using a learning algorithm, i.e., inputted into a predictive model, as in the examples provided herein (Examples 1-3). The parameters of the analytic process may be those disclosed herein or those derived using the guidelines described herein. Learning algorithms such as linear discriminant analysis, recursive feature elimination, a prediction analysis of microarray, logistic regression, CART, FlexTree, LART, random forest, MART, or another machine learning algorithm are applied to the appropriate reference or training data to determine the parameters for analytical processes suitable for a AS response or non-response classification.

The analytic process may set a threshold for determining the probability that a sample belongs to a given class. The probability preferably is at least 50%, or at least 60% or at least 70% or at least 80% or higher.

In other embodiments, the analytic process determines whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

In general, the analytical process will be in the form of a model generated by a statistical analytical method such as a linear algorithm, a quadratic algorithm, a polynomial algorithm, a decision tree algorithm, a voting algorithm.

### Use of Reference/Training Datasets to Determine Parameters of Analytical Process

Using any suitable learning algorithm, an appropriate reference or training dataset is used to determine the parameters of the analytical process to be used for classification, i.e., develop a predictive model.

The reference or training dataset to be used will depend on the desired AS classification to be determined, e.g. responder or non-responder. The dataset may include data from two, three, four or more classes.

For example, to use a supervised learning algorithm to determine the parameters for an analytic process used to predict response to anti-TNFalpha therapy, a dataset comprising control and diseased samples is used as a training set. Alternatively, a supervised learning algorithm is to be used to develop a predictive model for AS disease therapy.

### Statistical Analysis

The following are examples of the types of statistical analysis methods that are available to one of skill in the art to aid in the practice of the disclosed methods. The statistical analysis may be applied for one or both of two tasks. First, these and other statistical methods may be used to identify preferred subsets of the markers and other indicia that will form a preferred dataset. In addition, these and other statistical methods may be used to generate the analytical process that will be used with the dataset to generate the result. Several of statistical methods presented herein or otherwise available in the art will perform both of these tasks and yield a model that is suitable for use as an analytical process for the practice of the methods disclosed herein.

In a specific embodiment, biomarkers and their corresponding features (e.g., expression levels or serum levels) are used to develop an analytical process, or plurality of analytical processes, that discriminate between classes of patients, e.g. responder and non-responder to anti-TNFalpha therapy. Once an analytical process has been built using these exemplary data analysis algorithms or other techniques known in the art, the analytical process can be used to classify a test subject into one of the two or more phenotypic classes (e.g. a patient predicted to respond to anti-TNFalpha therapy or a patient who will not respond). This is accomplished by applying the analytical process to a marker profile obtained from the test subject. Such analytical processes, therefore, have enormous value as diagnostic indicators.

The disclosed methods provide, in one aspect, for the evaluation of a marker profile from a test subject to marker profiles obtained from a training population. In some embodiments, each marker profile obtained from subjects in the training population, as well as the test subject, comprises a feature for each of a plurality of different markers. In some embodiments, this comparison is accomplished by (i) developing an analytical process using the marker profiles from the training population and (ii) applying the analytical process to the marker profile from the test subject. As such, the analytical process applied in some embodiments of the methods disclosed herein is used to determine whether a test AS patient is predicted to respond to anti-TNFalpha therapy or a patient who will not respond.

Thus, in some embodiments, the result in the above-described binary decision situation has four possible outcomes: (i) a true responder, where the analytical process indicates that the subject will be a responder to anti-TNFalpha therapy and the subject does in fact respond to anti-TNFalpha therapy during the definite time period (true positive, TP); (ii) false responder, where the analytical process indicates that the subject will be a responder to anti-TNFalpha therapy and the subject does not respond to anti-TNFalpha therapy during the definite time period (false positive, FP); (iii) true non-responder, where the analytical process indicates that the will not be a responder to anti-TNFalpha therapy and the subject does not respond to anti-TNFalpha therapy during the definite time period (true negative, TN); or (iv) false non-responder, where the analytical process indicates that the patient will not be a responder to anti-TNFalpha therapy and the subject does in fact respond to anti-TNFalpha therapy during the definite time period (false negative, FN).

Relevant data analysis algorithms for developing an analytical process include, but are not limited to, discriminant analysis including linear, logistic, and more flexible discrimination techniques (see, e.g., Gnanadesikan, 1977, Methods for Statistical Data Analysis of Multivariate Observations, New York: Wiley 1977, which is hereby incorporated by reference herein in its entirety); tree-based algorithms such as classification and regression trees (CART) and variants (see, e.g., Breiman, 1984, Classification and Regression Trees, Belmont, Calif.: Wadsworth International Group, which is hereby incorporated by reference herein in its entirety); generalized additive models (see, e.g., Tibshirani, 1990, Generalized Additive Models, London: Chapman and Hall, which is hereby incorporated by reference herein in its entirety); and neural networks (see, e.g., Neal, 1996, Bayesian Learning for Neural Networks, New York: Springer-Verlag; and Insua, 1998, Feedforward neural networks for nonparametric regression In: Practical Nonparametric and Semiparametric Bayesian Statistics, pp. 181-194, New York: Springer, which is hereby incorporated by reference herein in its entirety).

In a specific embodiment, a data analysis algorithm of the invention comprises Classification and Regression Tree (CART), Multiple Additive Regression Tree (MART), Prediction Analysis for Microarrays (PAM) or Random Forest analysis. Such algorithms classify complex spectra from biological materials, such as a blood sample, to distinguish subjects as normal or as possessing biomarker expression levels characteristic of a particular disease state. In other embodiments, a data analysis algorithm of the invention comprises ANOVA and nonparametric equivalents, linear discriminant analysis, logistic regression analysis, nearest neighbor classifier analysis, neural networks, principal component analysis, quadratic discriminant analysis, regression classifiers and support vector machines.

While such algorithms may be used to construct an analytical process and/or increase the speed and efficiency of the application of the analytical process and to avoid investigator bias, one of ordinary skill in the art will realize that a computer-based device is not required to carry out the methods of using the predictive models of the present invention.

### Results of the CART Analysis

In one aspect of the present invention, the analyses of serum markers in patients diagnosed with AS was focused on significant relationships between biomarker baseline values and response to anti-TNFa therapy. In another aspect of the present invention, the analyses of the change in serum markers from baseline (prior to anti-TNFalpha therapy) to Week 4 after therapy in serum markers in patients diagnosed with AS was related to the clinical response or non-response of the patient at a later time (Week 14).

In a specific embodiment of the invention, it was found that the baseline concentration of leptin could be an initial classifier; for predicting the Week 14 outcome assessed as ASAS20 for the patients treated with golimumab. In an alternate embodiment, baseline osteocalin could be an initial classifier; for predicting the Week 14 outcome assessed as ASAS20 or as BASDAI for the patients treated with golimumab. This information can be used by physicians to determine who is benefiting from golimumab treatment, and just as important, to identify those patients are not benefiting from such treatment.

Alternatively, BASDAI was used as the clinical outcome component of the model. and TIMP-1 at baseline, osteocalcin at baseline, or change in complement component 3 was the initial marker for classification. in combination with changes in G-CSF when the TIMP-1 value was elevated, and Prostatic Acid phosphatase when the TIMP-1 value was below the cutoff plus a MCP-1 value below a cutoff value predicted the outcome at Week 14.

### Baseline Biomarkers Prediction of Response to Anti-TNFa Therapy.

When a predictive algorithm was built from datasets comprising only the baseline biomarkers serum concentration values and correlated with clinical response of an AS patient treated with an anti-TNF alpha therapeutic in more than one method of assessing clinical response, such as ASAS20 and BASDAI, the markers included leptin, TIMP-1, CD40 ligand, G-CSF, MCP-1, osteocalcin, PAP, and insulin.

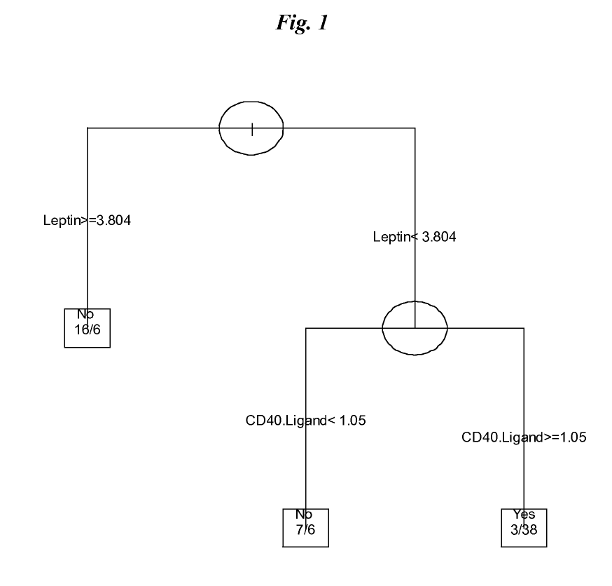

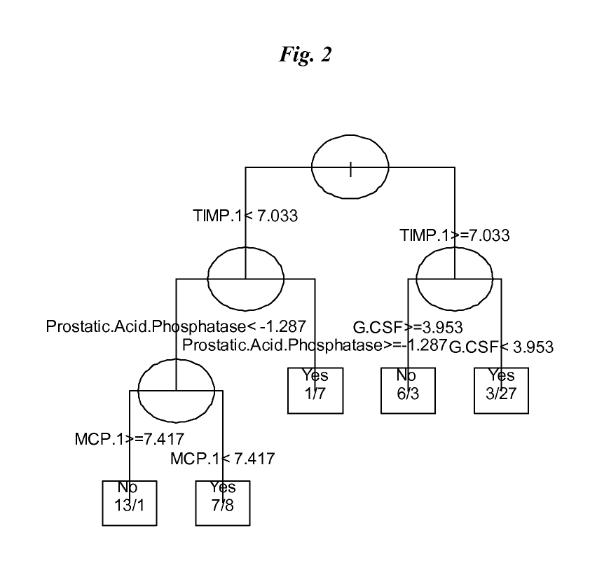

As demonstrated herein, analysis of biomarkers in serum obtained from AS patients at baseline (Week 0, prior to treatment), quantitated by a multiplexed assay, the best CART model included leptin as the initial classifier: subjects with leptin above 3.8 (log scale) are predicted to be non-responders; subjects with leptin below 3.8 are classified based on the secondary predictor of CD40 ligand (CD40 ligand above 1.05 predicted as responders, CD40 ligand below 1.05 predicted as non-responders) (FIG. 1). The model sensitivity was 86%, and model specificity was 88%. When the clinical measure was change from baseline to Week 14 in BASDAI and baseline biomarker data quantitated by multiplex different biomarkers became classifiers: TIMP-1, prostatic acid phosphatase, GCSF, and MCP-1 (FIG. 2) but the overall accuracy of the BASDAI model was similar to the ASAS20 model.

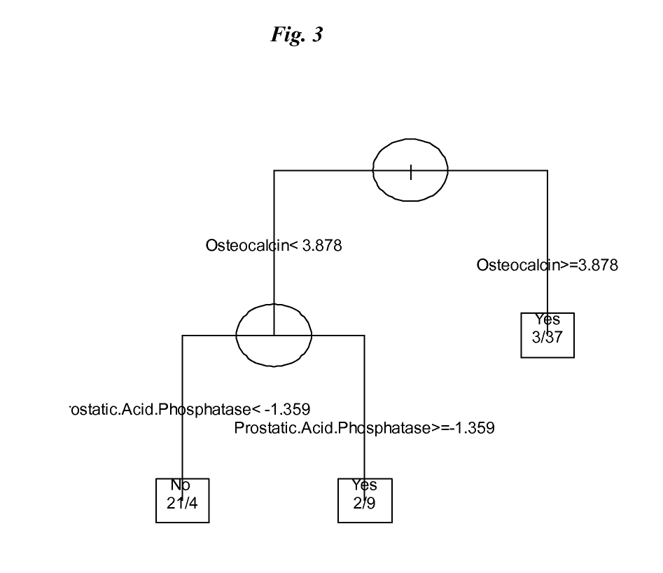

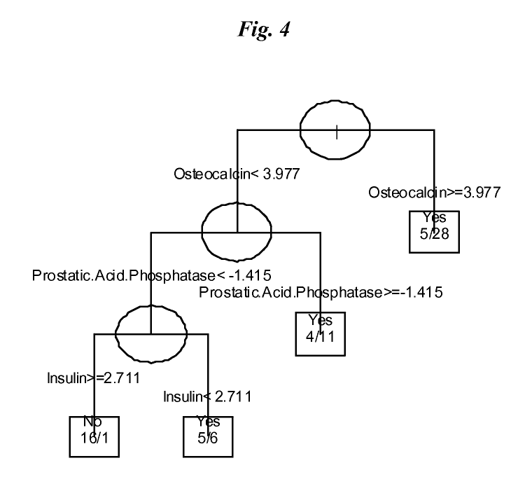

The analysis of biomarkers in serum obtained from AS patients at baseline (Week 0, prior to treatment), quantitated by both a multiplexed assay and individual EIA, the best CART model included osteocalcin as the initial classifier: subjects with osteocalcin above 3.878 (log scale) are predicted to be responders; subjects with osteocalcin below 3.878 are further classified based on prostatic acid phosphatase (FIG. 3). The model sensitivity was 90%, and model specificity was 84%. Thus, by using data from a multiplexed assay in addition to individual EIA assays and correlating the results to either BASDAI and ASAS20 produced models which both included osteocalcin and prostatic acid phosphatase as classifiers. The BASDAI-based model incorporated insulin as one additional classifier. The model accuracy was 61/76 (80%) for prediction of BASDAI clinical response (FIG. 4).

These results suggest that baseline levels of biomarkers can be measured prior to treatment by a physician to identify which patients treated with golimumab will respond or not respond to the treatment.

### Biomarker Change as Early Predictor of Outcome

Biomarker change from baseline serum levels at Week 4 in AS patient found to correlate with clinical response in more than one method of assessing clinical response, such as ASAS20 and BASDAI, include: leptin, VEGF, complement 3, ICAM-1, and ferritin.

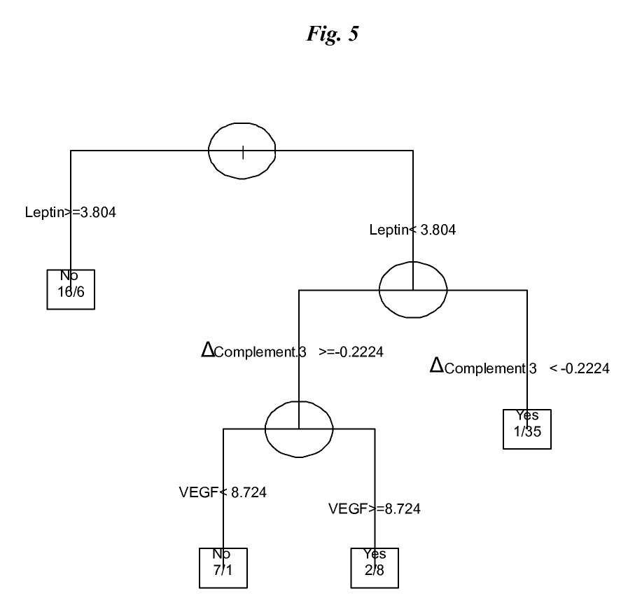

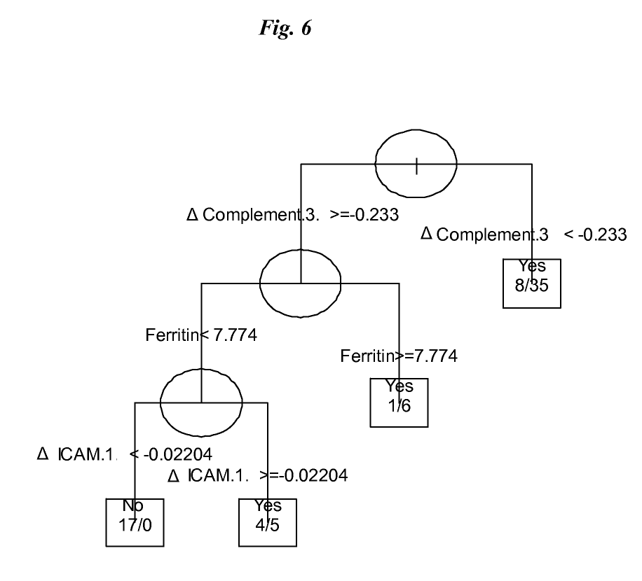

For analysis of biomarkers in serum obtained from AS patients at baseline and Week 4 quantitated by multiplex only, the biomarker model uses leptin as the initial classifier: subjects with leptin above 3.8 (log scale) are predicted to be non-responders; subjects with leptin below 3.8 are classified based on two additional classifiers: i) change in complement 3, and ii) VEGF (FIG. 5). Model sensitivity was 92%, and model specificity was 81%. When the clinical measure was change from baseline to Week 14 in BASDAI, the overall accuracy was similar to the ASAS20 model, change in complement component 3 was the initial classifier followed by two subclassifications using baseline ferritin followed by change in ICAM-1 (FIG. 6).

The specific examples described herein for generating an algorithm useful for predicting the response or non-response of an AS patient to anti-TNFalpha therapy indicate that multiple markers are correlative of AS processes and the quantitative interpretation of each particular biomarker in diagnosing or predicting response to therapy has not been heretofore well established. The applicants have demonstrated that an algorithm can be generated using a sampling of patient data based on specific markers defined. In one method of using the markers of the invention, a computer assisted device is used to capture patient data and perform the necessary analysis. In another aspect, the computer-assisted device or system may use the data presented herein as a “training data set” in order to generate the classifier information required to apply the predictive analysis.

### Instruments, Reagents and Kits for Performing the Analysis

The measurement of the serum biomarkers for predicting response of a diagnosed AS patient to anti-TNF therapy may be performed in a clinical or research laboratory or a centralized laboratory in a hospital or non-hospital location using standard immunochemical and biophysical methods as described herein. The marker quantitation may be performed at the same time as e.g. other standard measures such as WBC count, platelets, and ESR. The analysis may be performed individually or in batches using commercial kits, or using multiplexed analysis on individual patient samples.

In one aspect of the invention, individual and sets of reagents are used in one or more steps to determine relative or absolute amounts of a biomarker, or panel or biomarkers, in a patient's sample. The reagents may be used to capture the biomarker, such as an antibody immunospecific for a biomarker, which forms a ligand biomarker pair detectable by an indirect measurement such as enzyme-linked immunospecific assay. Either single analyte EIA or multiplexed analysis can be performed. Multiplexed analysis is a technique by which multiple, simultaneous EIA-based assays can be performed using a single serum sample. One platform useful to quantify large numbers of biomarkers in a very small sample volume is the xMAP® technology used by Rules Based Medicine in Austin, Tex. (owned by the Luminex Corporation), which performs up to 100 multiplexed, microsphere-based assays in a single reaction vessel by combining optical classification schemes, biochemical assays, flow cytometry and advanced digital signal processing hardware and software. In the technology, multiplexing is accomplished by assigning each analyte-specific assay a microsphere set labeled with a unique fluorescence signature. Multiplexed assays are analyzed in a flow device that interrogates each microsphere individually as it passes through a red and green laser. Alternatively, methods and reagents are used to process the sample for detection and possible quantitation using a direct physical measurement such as mass, charge, or a combination such as by SELDI. Quantitative mass spectrometric multiple reaction monitoring assays have also been developed such as those offered by NextGen Sciences (Ann Arbor, Mich.).

According to one aspect of the invention, therefore, the detection of biomarkers for evaluation of AS status entails contacting a sample from a subject with a substrate, e.g., a probe, having capture reagent thereon, under conditions that allow binding between the biomarker and the reagent, and then detecting the biomarker bound to the adsorbent by a suitable method. One method for detecting the marker is gas phase ion spectrometry, for example, mass spectrometry. Other detection paradigms that can be employed to this end include optical methods, electrochemical methods (voltametry, amperometry or electrochemiluminescent techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry), and enzyme-coupled colorimetric or fluorescent methods.

Specimens from patients may require processing prior to applying the detecting method to the processed specimen or sample such as but not limited to methods to concentrate, purify, or separate the marker from other components of the specimen. For example a blood sample is typically treated with an anticoagulant and the cellular components and platelets removed prior to being subjected to methods of detecting analyte concentration. Alternatively, the detecting may be accomplished by a continuous processing system which may incorporate materials or reagents to accomplish such concentrating, separating or purifying steps. In one embodiment the processing system includes the use of a capture reagent. One type of capture reagent is a “chromatographic adsorbent,” which is a material typically used in chromatography. Chromatographic adsorbents include, for example, ion exchange materials, metal chelators, immobilized metal chelates, hydrophobic interaction adsorbents, hydrophilic interaction adsorbents, dyes, simple biomolecules (e.g., nucleotides, amino acids, simple sugars and fatty acids), mixed mode adsorbents (e.g., hydrophobic attraction/electrostatic repulsion adsorbents). A “biospecific” capture reagent is a capture reagent that is a biomolecule, e.g., a nucleotide, a nucleic acid molecule, an amino acid, a polypeptide, a polysaccharide, a lipid, a steroid or a conjugate of these (e.g., a glycoprotein, a lipoprotein, a glycolipid). In certain instances the biospecific adsorbent can be a macromolecular structure such as a multiprotein complex, a biological membrane or a virus. Illustrative biospecific adsorbents are antibodies, receptor proteins, and nucleic acids. A biospecific adsorbent typically has higher specificity for a target analyte than a chromatographic adsorbent.

The detection and quantitation of the biomarkers according to the invention can thus be enhanced by using certain selectivity conditions, e.g., adsorbents or washing solutions. A wash solution refers to an agent, typically a solution, which is used to affect or modify adsorption of an analyte to an adsorbent surface and/or to remove unbound materials from the surface. The elution characteristics of a wash solution can depend, for example, on pH, ionic strength, hydrophobicity, degree of chaotropism, detergent strength, and temperature.

In one aspect of the present invention, a sample is analyzed in a multiplexed manner meaning that the processing of markers from a patient samples occurs substantially simultaneously. In one aspect, the sample is contacted by a substrate comprising multiple capture reagents representing unique specificity. The capture reagents are commonly immunospecific antibodies or fragments thereof. The substrate may be a single component such as a “biochip,” a term that denotes a solid substrate, having a generally planar surface, to which a capture reagent(s) is attached, or the capture reagents may be segregated among a number of substrates, as for example bound to individual spherical substrates (beads). Frequently, the surface of a biochip comprises a plurality of addressable locations, each of which has the capture reagent bound there. A biochip can be adapted to engage a probe interface and, hence, function as a probe in gas phase ion spectrometry preferably mass spectrometry. Alternatively, a biochip of the invention can be mounted onto another substrate to form a probe that can be inserted into the spectrometer. In the case of the beads, the individual beads may be partitioned or sorted after exposure to the sample for detection.

A variety of biochips are available for the capture and detection of biomarkers, in accordance with the present invention, from commercial sources such as Ciphergen Biosystems (Fremont, Calif.), Perkin Elmer (Packard BioScience Company (Meriden Conn.), Zyomyx (Hayward, Calif.), and Phylos (Lexington, Mass.), GE Healthcare, Corp. (Sunnyvale, Calif.). Exemplary of these biochips are those described in U.S. Pat. No. 6,225,047, supra, and U.S. Pat. No. 6,329,209 (Wagner et al.), and in WO 99/51773 (Kuimelis and Wagner), WO 00/56934 (Englert et al.) and particularly those which use electrochemical and electrochemiluminescence methods of detecting the presence or amount of an analyte marker in a sample such as those multi-specific, multi-array taught in Wohlstadter et al., WO98/12539 and U.S. Pat. No. 6,066,448.

A substrate with biospecific capture and/or detection reagents is contacted with the sample, containing e.g. serum, for a period of time sufficient to allow biomarker that may be present to bind to the reagent. In one embodiment of the invention, more than one type of substrate with biospecific capture or detection reagents thereon is contacted with the biological sample. After the incubation period, the substrate is washed to remove unbound material. Any suitable washing solutions can be used; preferably, aqueous solutions are employed.

Biomarkers bound to the substrates are to be detected after desorption directly by using a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined. Such methods may be used to discovery biomarkers and, in some instances for quantitation of biomarkers.

In another embodiment, the method of the invention is a microfluidic device capable of miniaturized liquid sample handling and analysis device for liquid phase analysis as taught in, for example, U.S. Pat. No. 5,571,410 and USRE36350, useful for detecting and analyzing small and/or macromolecular solutes in the liquid phase, optionally, employing chromatographic separation means, electrophoretic separation means, electrochromatographic separation means, or combinations thereof The microfluidic device or “microdevice” may comprise multiple channels arranged so that analyte fluid can be separated, such that biomarkers may be captured, and, optionally, detected at addressable locations within the device (U.S. Pat. No. 5,637,469, U.S. Pat. No. 6,046,056 and U.S. Pat. No. 6,576,478).

Data generated by detection of biomarkers can be analyzed with the use of a programmable digital computer. The computer program analyzes the data to indicate the number of markers detected and the strength of the signal. Data analysis can include steps of determining signal strength of a biomarker and removing data deviating from a predetermined statistical distribution. For example, the data can be normalized relative to some reference. The computer can transform the resulting data into various formats for display, if desired, or further analysis.

### Artificial Neural Network

In some embodiments, a neural network is used. A neural network can be constructed for a selected set of markers. A neural network is a two-stage regression or classification model. A neural network has a layered structure that includes a layer of input units (and the bias) connected by a layer of weights to a layer of output units. For regression, the layer of output units typically includes just one output unit. However, neural networks can handle multiple quantitative responses in a seamless fashion.

In multilayer neural networks, there are input units (input layer), hidden units (hidden layer), and output units (output layer). There is, furthermore, a single bias unit that is connected to each unit other than the input units. Neural networks are described in Duda et al., 2001, Pattern Classification, Second Edition, John Wiley &amp; Sons, Inc., New York; and Hastie et al., 2001, The Elements of Statistical Learning, Springer-Verlag, New York

The basic approach to the use of neural networks is to start with an untrained network, present a training pattern, e.g., marker profiles from patients in the training data set, to the input layer, and to pass signals through the net and determine the output, e.g., the prognosis of the patients in the training data set, at the output layer. These outputs are then compared to the target values, e.g. actual outcomes of the patients in the training data set; and a difference corresponds to an error. This error or criterion function is some scalar function of the weights and is minimized when the network outputs match the desired outputs. Thus, the weights are adjusted to reduce this measure of error. For regression, this error can be sum-of-squared errors. For classification, this error can be either squared error or cross-entropy (deviation). See, e.g., Hastie et al., 2001, The Elements of Statistical Learning, Springer-Verlag, New York.

Three commonly used training protocols are stochastic, batch, and on-line. In stochastic training, patterns are chosen randomly from the training set and the network weights are updated for each pattern presentation. Multilayer nonlinear networks trained by gradient descent methods such as stochastic back-propagation perform a maximum-likelihood estimation of the weight values in the model defined by the network topology. In batch training, all patterns are presented to the network before learning takes place. Typically, in batch training, several passes are made through the training data. In online training, each pattern is presented once and only once to the net.

In some embodiments, consideration is given to starting values for weights. If the weights are near zero, then the operative part of the sigmoid commonly used in the hidden layer of a neural network (see, e.g., Hastie et al., 2001, The Elements of Statistical Learning, Springer-Verlag, New York) is roughly linear, and hence the neural network collapses into an approximately linear model. In some embodiments, starting values for weights are chosen to be random values near zero. Hence the model starts out nearly linear, and becomes nonlinear as the weights increase. Individual units localize to directions and introduce nonlinearities where needed. Use of exact zero weights leads to zero derivatives and perfect symmetry, and the algorithm never moves. Alternatively, starting with large weights often leads to poor solutions.

Since the scaling of inputs determines the effective scaling of weights in the bottom layer, it can have a large effect on the quality of the final solution. Thus, in some embodiments, at the outset all expression values are standardized to have mean zero and a standard deviation of one. This ensures all inputs are treated equally in the regularization process, and allows one to choose a meaningful range for the random starting weights. With standardization inputs, it is typical to take random uniform weights over the range −0.7, +0.7.

A recurrent problem in the use of networks having a hidden layer is the optimal number of hidden units to use in the network. The number of inputs and outputs of a network are determined by the problem to be solved. For the methods disclosed herein, the number of inputs for a given neural network can be the number of markers in the selected set of markers.

The number of outputs for the neural network will typically be just one: yes or no. However, in some embodiment more than one output is used so that more than just two states can be defined by the network.

Software used to analyze the data can include code that applies an algorithm to the analysis of the signal to determine whether the signal represents a peak in a signal that corresponds to a biomarker according to the present invention. The software also can subject the data regarding observed biomarker signals to classification tree or ANN analysis, to determine whether a biomarker or combination of biomarker signals is present that indicates patient's disease diagnosis or status.

Thus, the process can be divided into the learning phase and the classification phase. In the learning phase, a learning algorithm is applied to a data set that includes members of the different classes that are meant to be classified, for example, data from a plurality of samples from patients diagnosed as AS and who respond to anti-TNFa therapy and data from a plurality of samples from patients with a negative outcome, AS patients who did not respond to anti-TNFa therapy. The methods used to analyze the data include, but are not limited to, artificial neural network, support vector machines, genetic algorithm and self-organizing maps and classification and regression tree analysis. These methods are described, for example, in WO01/31579, May 3, 2001 (Barnhill et al.); WO02/06829, Jan. 24, 2002 (Hitt et al.) and WO02/42733, May 30, 2002 (Paulse et al.). The learning algorithm produces a classifying algorithm keyed to elements of the data, such as particular markers and specific concentrations of markers, usually in combination, that can classify an unknown sample into one of the two classes, e.g. responder on non-responder. The classifying algorithm is ultimately used for predictive testing.

Software, both freeware and proprietary software, is readily available to analyze patterns in data, and to devise additional patterns with any predetermined criteria for success.

### Kits

In another aspect, the present invention provides kits for determining which AS patients will respond or not respond to treatment with an anti-TNFa agent, such as golimumab, which kits are used to detect serum markers according to the invention. The kits screen for the presence of serum markers and combinations of markers that are differentially present in AS patients.

In one aspect, the kit contains a means for collecting a sample, such as a lance or piercing tool for causing a “stick” through the skin. The kit may, optionally, also contain a probe, such as a capillary tube, for collecting blood from the stick.

In one embodiment, the kit comprises a substrate having one or more biospecific capture reagents for binding a marker according to the invention. The kit may include more than type of biospecific capture reagents, each present on the same or a different substrate.

In a further embodiment, such a kit can comprise instructions for suitable operational parameters in the form of a label or separate insert. For example, the instructions may inform a consumer how to collect the sample or how to empty or wash the probe. In yet another embodiment the kit can comprise one or more containers with biomarker samples, to be used as standard(s) for calibration.

In the method of using the algorithm of the invention for predicting the response of an AS patient to anti-TNF therapy, blood or other fluid is acquired from the patient prior to anti-TNF therapy and at specified periods after therapy is initiated. The blood may be processed to extract a serum fraction or be used whole. The blood or serum samples may be diluted, for example 1:2, 1:5, 1:10, 1:20, 1:50, or 1:100, or used undiluted. In one format, the serum or blood sample is applied to a prefabricated test strip or stick and incubated at room temperature for a specified period of time, such as 1 min, 5 min, 10 min, 15, min, 1 hour, or longer. After the specified period of time the for the assay; the samples and the result are readable directly from the strip. For example, the results appear as varying shades of colored or gray bands, indicating a concentration range of one or more markers. The test strip kit will provide instructions for interpreting the results based on the relative concentrations of the one or more markers. Alternatively, a device capable of detecting the color saturation of the marker detection system on the strip can be provide, which device may optionally provide the results of the test interpretation based on the appropriate diagnostic algorithm for that series of markers.

### Methods of Using the Invention

The invention provides a method of predicting responsiveness to therapy with an anti-TNFalpha agent, such as golimumab, by analyzing detected biomarkers in a patient diagnosed with AS. In the method of the invention, a patient is first diagnosed with AS by an experienced professional using subjective and objective criteria.

Ongoing investigation of the pathogenesis of AS are focused on identifying initiating factors, downstream events, mediators of inflammation, and regulators of the process. It has been estimated that approximately 90% of the risk of developing AS is heritable. The most powerful of the genetic risk factors is related to the HLA-B27 molecule. Given the important role that HLA-B27 plays in risk, several possible mechanisms have been proposed. However, despite the intense interest and active investigation, there is yet no general consensus on how HLA-B27 contributes to disease susceptibility. The role of environmental factors remains elusive, as does the understanding of the propensity of AS to involve attachment of ligaments and tendons to bone (entheses) or the involvement of the sacroiliac joints.

The primary clinical features of AS include inflammatory back pain caused by sacroiliitis, inflammation at other locations in the axial skeleton, peripheral arthritis, enthesitis, and anterior uveitis. Structural changes are caused mainly by osteoproliferation rather than osteodestruction. Syndesmophytes and ankylosis are the most characteristic features of this disease. The characteristic symptoms of AS are low-back pain, buttock pain, limited spinal mobility, hip pain, shoulder pain, peripheral arthritis, and enthesitis. Neurological symptoms can occur with cord or spinal nerve compression resulting from several complications of the disease. Vertebral fractures can develop in patients with ankylosed spines with minimal or no traumatic injury. The most common fracture site is at the C5-6 interspace. Clinically significant atlantoaxial subluxation can occur in up to 21% of patients with AS and can lead to spinal cord compression. Cauda equina syndrome is a rare complication of longstanding AS; its pathogenesis is poorly understood and includes inflammation, arachnoiditis, mechanical stretching, compression of the nerve roots, demyelination, and ischemia.

### Clinical Assessment Methods

The diagnosis of AS is made from a combination of clinical features and evidence of sacroiliitis by some imaging technique defined by the 1984 Modified New York Criteria (van der Linden S, Valkenburg H A, Cats A: Evaluation of diagnostic criteria for ankylosing spondylitis. A proposal for modification of the New York criteria. Arthritis Rheum 27:361-368, 1984). Laboratory markers of disease, such as the erythrocyte sedimentation rate (ESR) and C-reactive protein (CRP) levels has been shown to be unhelpful in assessing disease activity or monitoring the response to treatment (Spooorenberg A et al. 1999 J Rheumatol 26:980-4).

The clinical criteria are: 1) low-back pain and stiffness of more than 3 months' duration that improves with exercise but is not relieved by rest; 2) limitation of motion of the lumbar spine in both the sagittal and frontal (coronal) planes; and 3) limitation of chest expansion relative to normal values corrected for age and sex. The radiological criteria are sacroiliitis Grade 2 or higher bilaterally, or Grade 3 or higher unilaterally. The radiographic grading of sacroiliitis consists of 5 grades: Grade 0 is a normal spine; Grade 1 indicates suspicious changes; Grade 2 indicates sclerosis with some erosion; Grade 3 indicates severe erosions, pseudodilatation of the joint space, and partial ankylosis; and Grade 4 denotes complete ankylosis. Definite AS is present when 1 radiological criterion is associated with at least 1 clinical criterion. Probable AS is considered if there are three clinical criteria present or radiologic criteria exist with no signs or symptoms to satisfy the clinical criteria. Clinical Grades may be used as part of the data set for generating a predictive algorithm for response to therapy.

Once the diagnosis of AS is established, the physician generally monitors clinical outcomes longitudinally in order to identify patients at risk of worsening disease. The ankylosing Spondylitis Assessment Study Group (ASAS) has defined a number of core parameters of the disease for management. Pain in AS patients is usually confined to the back, but extra-axial sites can be the main focus of pain-relieving therapy in patients with peripheral disease manifestations. A single 100 mm horizontal visual analog scale (VAS) is used to measure nocturnal and general spinal pain. In AS patients treated with anti-TNF therapy, the ASAS has developed response criteria. Several of these criteria are outlined below or can be obtained by contacting the American Society or Rhuematologists.

ASAS20 reflects the improvement by 20% of several criteria used to generate a “score” (Anderson J J et al. 2001 Arthritis Rheum 44: 1876-1886). The ASAS improvement criteria define a positive response to treatment as, firstly, a 20% relative improvement and, secondly, 10 units of absolute improvement in three of four domains (inflammation, function, patient perception of pain and patient global health, with no worsening in the fourth domain).

BASDAI (Bath Ankylosing Spondylitis Disease Activity Index) defines the inflammatory activity in a patient with AS. Inflammation can be evaluated clinically by assessing the degree of discomfort and morning stiffness experienced by the patient. The BASDAI is a self-administered index with each question being framed in a 100 mm VAS (range 0-100, where 0=no stiffness and 100=very severe stiffness). The score has been shown to be sensitive to change with treatment.

BASMI (Bath Ankylosing Spondylitis Metrology Index) is a quantitative, physician assessed measure of the spinal mobility limitations experienced by a patient with AS. BASMI is a validated index consisting of five clinical measurements including cervical rotation, tragus-to-wall distance, lateral spine flexion, lumbar flexion and intermalleolar distance, which reflects axial segmental involvement. The BASMI has been shown to demonstrate good inter-observer reliability; however, the BASMI cannot distinguish physical limitations as a consequence of acute inflammation from those caused by chronic disease damage. Although there are no published longitudinal studies demonstrating the progression of BASMI over the lifespan of a patient, it is assumed that a patient's BASMI score would increase gradually over time as the AS patient develops progressive disease. The correlation of the BASMI with spinal radiographs have, in some cases, demonstrated a significant correlation with the presence of radiographic damage.

BASFI (Bath Ankylosing Spondylitis Functional Index) uses physical function measures to assess the degree of limitation in a patient's ability to carry out everyday tasks. Physical function is measured using the BASFI and the Dougados Functional Index (DFI). The BASFI, however, is the measure that is most widely used both in clinical practice and in clinical trials.

It will be recognized that the clinical indices described herein are part of the patient data set and can be assigned a numerical score.

### Failure of Previous Therapy

The ASAS has prepared a consensus statement on need for anti-TNF therapy in AS (Braun et al 2003 Annals Rheumatic Diseases 62:817-824). For all three presentations of AS; axial disease, peripheral arthritis, and enthesitis, treatment failure was defined as a trial of at least three months of standard NSAID treatment. Before starting anti-TNF therapy, patients must have had an adequate therapeutic trial of at least two NSAIDs based on the use of maximal recommended or tolerated anti-inflammatory doses, unless these drugs are contraindicated.

The failure of NSAID treatment is required for all three presentations: axial disease, peripheral arthritis, and enthesitis:

For symptomatic axial disease, no additional treatment is required before initiation of anti-TNF therapy

For symptomatic peripheral arthritis, failure of intra-articular corticosteroid treatment (at least two injections) is normally required in oligoarthritis. Unless contraindicated or not tolerated, standard DMARD treatment with sulfasalazine at maximally tolerated doses up to 3 g/day should be prescribed for four months

For symptomatic enthesitis, an adequate therapeutic trial of at least two local steroid injections is normally required, as long as these injections are not contraindicated.

Suitability for TNFa Therapy

Anti-TNFalpha agents have been commercially available, such as infliximab, and used to treat AS for several years. The anti-TNFα agents have been shown to result in dramatic improvement in ankylosing spondylitis, ameliorating the different symptoms of the disease, as well as improving the quality of life. An AS patient may be considered a candidate for anti-TNF alpha therapy based on additional criteria beyond the clinical assessment and, optionally, failure to respond to alternative therapy such as NSAIDs and physiotherapy, sulfasalzine or methotrexate or bisphosphonates.

### Patient Management

In the method of the invention for predicting or assessing early responsiveness to anti-TNF therapy, prior to initiation of anti-TNF therapy, at a “baseline visit”, a baseline or “Week 0” sample is acquired from the patient to be treated with anti-TNF therapy. The sample may be any tissue which can be evaluated for the biomarkers associated with the method of the invention. In one embodiment the sample is a fluid selected from the group consisting of a fluid selected from the group consisting of blood, serum, urine, semen and stool. In a particular embodiment, the sample is a serum sample which is obtained from patient's blood drawn by a standard method of direct venipuncture or via an intravenous catheter.

In addition, at the baseline visit, information on patient's demographics and history of disease with AS will be recorded on a standarized form or case report form. Data such as time since patient's diagnosis, previous treatment history, concomitant medications, C-reactive protein (CRP) level and an assessment of disease activity (ie BASDAI, BASMI) will be recorded.

The patient receives the first dose of anti-TNF therapy at the time of the baseline visit or within 24-48 hours. At the time of the baseline visit, the patient is scheduled for a Week 4 visit.

At the 4-week visit, approximately 28 days after initial administration of anti-TNFa therapy, a second patient sample is acquired, preferably using the same protocol and route as for the baseline sample. The patient is examined and other indices, imaging, or information may be performed or monitored as proscribed by the health care professional or study design as indicated. The patient is scheduled for subsequent visits, such as a Week 8, Week 12, Week 14, Week 28, etc. visit for the purposes of performing assessment of disease using the such criteria as set forth by the ASAS and BASDAI and for the acquisition of patient samples for biomarker evaluation.

At any or the above times prior to, during, or following treatment, other parameters and markers may be assessed in the patient's sample or other fluid or tissue samples acquired from the patient. These may include standard hematological parameters such as hemoglobin content, hematocrit, red cell volume, mean red cell diameter, erythrocyte sedimentation rate (ESR), and the like. Other markers may which have been determined useful in assessing the presence of AS may be quantitated in some or all of the patient's sample, such as, CRP (Spoorenberg A et al. 1999. J Rheumatol 26: 980-984) and IL-6, and markers of cartilage degradation such as serum Type 1 N-telopeptides (NTX), urinary type II collagen C-telopeptides (urinary CTX-II) and serum matrix metalloprotease 3 (MMP3, stromelysin 1) (See US20070172897).

Additional inflammation-related markers that may be of use in assessing the response to treatment may be inflammatory cytokines, such as IL-8, or IL-1, inflammatory chemokines, such as ENA-78/CXCL5, RANTES, MIP-1β; Angiogenesis associated proteins (EGF, VEGF); additional proteases such as MMP-9, TIMP-1; molecules acting on the cellular immune system (TH-1) such as IFNγ, IL-12p40, IP-10; and molecules acting on the humoral immune system (TH-2), including IL-4 and IL-13; growth factors such as FGF basic; general markers of Inflammation, including myeloperoxidase; and adhesion related molecules, such as ICAM-1.

The medical professional's clinical judgment of response should not be negated by the test result. However, the test could aid in making the decision to discontinue treatment with golimumab. In a test in which the prediction model (algorithm) has 90% sensitivity and 60% specificity, where 50% of the patients display a clinical response and 50% do not display assessment scores or evaluations consistent with a clinical response. This would mean: of the responders, 45% would be identified correctly as responders (5 would be reported as likely non-responders) and 30% or non-responders would be identified correctly as non-responders (20% would be classified as likely responders). Thus, overall benefit is that 60% of all true non-responders could be spared an unnecessary therapy or discontinued from therapy at an early timepoint (Week 4). The 5% false-negative “responders” (identified as likely non-responders) would have been treated, and as with all patients, their response would be judged clinically before making the decision to continue or discontinue treatment at Week 14 or later. The 20% false-negative “non-responders” (identified as possible responders) would have to be judged clinically, and would take the usual time to make the decision to discontinue treatment.

### EXAMPLE 1

### Sample Collection and Analysis

Serum samples were obtained and evaluated from patients enrolled in Centocor Protocol C0524T09, a multicenter, randomized, double-blind, placebo-controlled, 3-arm study. The three groups consist of a placebo and two dose levels of anti-TNFa Mab treatment; golimumab 50 mg, or golimumab 100 mg administered as SC injections every 4 weeks in patients with active Ankylosing Spondylitis. Primary efficacy assessments were made at week 14 and week 24. The serum samples for the biomarker study were collected from 100 patients at baseline (Week 0), Week 4, and Week 14.

The sera were analyzed for biomarkers using commercially available assays employing either a multiplex analysis performed by Rules Based Medicine (Austin, Tex.), or single analyte ELISA. All samples were stored at −80° C. until tested. The samples were thawed at room temperature, vortexed, spun at 13,000×g for 5 minutes for clarification and 150 uL was removed for antigen analysis into a master microtiter plate. Using automated pipetting, an aliquot of each sample was introduced into one of the capture microsphere multiplexes of the analytes. These mixtures of sample and capture microspheres were thoroughly mixed and incubated at room temperature for 1 hour. Multiplexed cocktails of biotinylated, reporter antibodies for each multiplex were used and detected using streptavidin-phycoerythrin. Analysis was performed in a Luminex 100 instrument and the resulting data stream was interpreted using proprietary data analysis software developed at Rules-Based Medicine and licensed to Qiagen Instruments. For each multiplex, both calibrators and controls were run. Testing results were determined first for the high, medium and low controls for each multiplex to ensure proper assay performance. Unknown values for each of the analytes localized in a specific multiplex were determined using 4 and 5 parameter, weighted and non-weighted curve fitting algorithms included in the data analysis package. At each timepoint, a total of 92 protein biomarkers were assayed (Table 1).

TABLE 1

| | | Swiss-Prot |
|---|---|---|
| Human Antigen | Units | Accession # |
| Adiponectin | ug/mL | Q15848 |
| Alpha-1 Antitypsin | mg/mL | P07758 |
| Alpha-2 Macroglobulin | mg/mL | P01023 |
| Alpha-Fetoprotein | ng/mL | P02771 |
| Apolipoprotein A-1 | mg/mL | P02647 |
| Apolipoprotein CIII | ug/mL | P02656 |
| Apolipoprotein H | ug/mL | P02749 |
| Beta 2-Microglobulin | ug/mL | P01884 |
| Brain-Derived Neurotrophic Factor (BDNF) | ng/mL | P23560 |
| Calcitonin | pg/mL | P01258 |
| Cancer Antigen 125 | U/mL | Q14596 |
| Cancer Antigen 19-9 | U/mL | Q9BXJ9 |
| Carcinoembryonic Antigen | ng/mL | P78448 |
| CD40 | ng/mL | P25942 |
| CD40 Ligand | ng/mL | P29965 |
| Complement component 3 | mg/mL | P01024 |
| C-Reactive Protein | ug/mL | P02741 |
| Creatine Kinase MB - Brain | ng/mL | P12277 |
| ENA-78 | ng/mL | P42830 |
| (Epithelial Neutrophil Activating Peptide 78) | | |
| Endothelin | pg/mL | P05305 |
| ENRAGE | ng/mL | P80511 |
| Eotaxin | pg/mL | P51671 |
| Epidermal Growth Factor | pg/mL | P01133 |
| Erythropoietin | pg/mL | P01588 |
| Factor VII | ng/mL | P08709 |
| Fatty Acid Binding Protein | ng/mL | P05413 |
| Ferritin - Heavy | ng/mL | P02794 |
| FGF-basic | pg/mL | P09038 |
| Fibrinogen alpha chain | mg/mL | P02671 |
| G-CSF | pg/mL | P09919 |
| Glutathione S-Transferase alpha | ng/mL | P08263 |
| GM-CSF | pg/mL | P04141 |
| Growth Hormone | ng/mL | P01241 |
| Haptoglobin | mg/mL | P00738 |
| ICAM-1 (Intercellular Adhesion Molecule 1) | ng/mL | P05362 |
| IFN gamma | pg/mL | P01579 |
| IgA | mg/mL | na |
| IgE | ng/mL | na |
| IGF-1 | ng/mL | P05019 |
| IgM | mg/mL | na |
| IL-1 receptor antagonist | pg/mL | Q9UBH0 |
| IL-10 | pg/mL | P22301 |
| IL-12 p40 | ng/mL | P29460 |
| IL-12 p70 | pg/mL | P29459 |
| IL-13 | pg/mL | P35225 |
| IL-15 | ng/mL | P40933 |
| IL-16 | pg/mL | Q14005 |
| IL-17 (IL17A) | pg/mL | Q16552 |
| IL-18 | pg/mL | Q14116 |
| IL-1alpha | ng/mL | P01583 |
| IL-1beta | pg/mL | P01584 |
| IL-2 | pg/mL | P01585 |
| IL-23 p19 | ng/mL | Q9NPF7 |
| IL-3 | ng/mL | P08700 |
| IL-4 | pg/mL | P05112 |
| IL-5 | pg/mL | P05113 |
| IL-6 | pg/mL | P05231 |
| IL-7 | pg/mL | P13232 |
| IL-8 | pg/mL | P10145 |
| Insulin | uIU/mL | P01308 |
| Leptin | ng/mL | P41159 |
| Lipoprotein (a) | ug/mL | P08519 |
| Lymphotactin | ng/mL | P47992 |
| MCP-1 (Monocyte Chemotactic Protein 1) | pg/mL | P13500 |
| MDC (Macrophage-Derived Chemokine) | pg/mL | O00626 |
| MIP-1 alpha (Macrophage Inflammatory | pg/mL | P10147 |
| Protein 1 alpha) | | |
| MIP-1 beta (Macrophage Inflammatory Protein | pg/mL | P13236 |
| 1 beta) | | |
| MMP-2 (Matrix Metalloproteinase 2) | ng/mL | P08253 |
| MMP-3 (Matrix Metalloproteinase 3) | ng/mL | P08254 |
| MMP-9 (Matrix Metalloproteinase 9) | ng/mL | P14780 |
| Myeloperoxidase | ng/mL | P05164 |
| Myoglobin | ng/mL | P02144 |
| PAI-1 | ng/mL | P05121 |
| PAPPA | mIU/mL | Q13219 |
| Prostate-Specific Antigen (PSA), Free | ng/mL | P07288 |
| Prostatic Acid Phosphatase (PAP) | ng/mL | P15309 |
| RANTES | ng/mL | P13501 |
| serum amyloid P component, (SA) | ug/mL | P02743 |
| SGOT (Serum Glutamic Oxaloacetic | ug/mL | P17174 |
| Transaminase) | | |
| SHBG | nmol/L | P04278 |
| Stem Cell Factor | pg/mL | P21583 |
| Thrombopoietin (TPO) | ng/mL | P40225 |
| Thyroid Stimulating Hormone (TSH) - alpha | uIU/mL | P01215 |
| Thyroxine Binding Globulin (TBG) | ug/mL | P05543 |
| TIMP-1 (Tissue Inhibitor of Metalloproteinase | ng/mL | P01033 |
| 1) | | |
| Tissue factor (coagulation factor III, | ng/mL | P13726 |
| thromboplastin) | | |
| TNF RII (Tumor Necrosis Factor Receptor 2) | ng/mL | Q92956 |
| TNF-alpha (Tumor Necrosis Factor alpha) | pg/mL | P01375 |
| TNF-beta (Tumor Necrosis Factor beta) | pg/mL | P01374 |
| VCAM-1 | ng/mL | P19320 |
| VEGF | pg/mL | P15692 |
| vWF (von Willebrand Factor) | ug/mL | P04275 |

Each of the 92 biomarkers has a lower limit of quantification (LOQ). The criterion for using a biomarker in the analysis required the biomarker to be above the limit of quantification in at least 20% of samples. Of the 92 biomarkers from the 300 samples, 63 (68%) met that criterion for inclusion in the analysis. An assessment of the distributions of each biomarker was made to determine whether a log transformation of that biomarker was warranted. This assessment was made without regard to treatment group. Overall, 60 of the 63 biomarkers in the analysis set were log 2 transformed. Table 2 identifies the biomarkers that were included in the final analysis, the LOQ, and whether log transformation was possible.

### Additional Baseline Biomarker Analysis

In addition to the Rules Based Medicine multiplex analysis, an additional set of serum biomarker data was generated using single EIA methods for certain markers not included in the multiplex test menu. The additional markers were combined with the multiplex biomarker data set to determine model accuracy based on combining the single and multiplex markers. These data were only included as part of the predictive models.

TABLE 2

| | | | #Samples | Log |
|---|---|---|---|---|
| | | | at LOQ | Trans- |
| Marker | Units | LOQ | (300 Total) | form |
| Adiponectin | ug/mL | 0.2 | 0 | TRUE |
| Alpha-1 Antitrypsin | mg/mL | 0.011 | 0 | TRUE |
| Alpha-2 Macroglobulin | mg/mL | 0.061 | 2 | TRUE |
| Alpha-Fetoprotein | ng/mL | 0.43 | 1 | TRUE |
| Apolipoprotein A1 | mg/mL | 0.0066 | 0 | TRUE |
| Apolipoprotein CIII | ug/mL | 2.7 | 0 | TRUE |
| Apolipoprotein H | ug/mL | 8.8 | 0 | TRUE |
| Beta-2 Microglobulin | ug/mL | 0.013 | 0 | TRUE |
| Brain-Derived Neurotrophic | ng/mL | 0.029 | 0 | TRUE |
| Factor | | | | |
| C Reactive Protein | ug/mL | 0.0015 | 0 | TRUE |
| Cancer Antigen 125 | U/mL | 4.2 | 5 | TRUE |
| Cancer Antigen 19-9 | U/mL | 0.25 | 26 | TRUE |
| Carcinoembryonic Antigen | ng/mL | 0.84 | 132 | TRUE |
| CD40 | ng/mL | 0.021 | 0 | TRUE |
| CD40 Ligand | ng/mL | 0.02 | 12 | FALSE |
| Complement 3 | mg/mL | 0.0053 | 0 | TRUE |
| EGF | pg/mL | 7.4 | 37 | TRUE |
| EN-RAGE | ng/mL | 0.25 | 0 | TRUE |
| ENA-78 | ng/mL | 0.076 | 0 | TRUE |
| Eotaxin | pg/mL | 41 | 29 | TRUE |
| Factor VII | ng/mL | 1 | 0 | TRUE |
| Ferritin | ng/mL | 1.4 | 0 | TRUE |
| Fibrinogen | mg/mL | 0.0098 | 78 | TRUE |
| G-CSF | pg/mL | 5 | 133 | TRUE |
| Glutathione S-Transferase | ng/mL | 0.4 | 1 | TRUE |
| Growth Hormone | ng/mL | 0.13 | 137 | TRUE |
| Haptoglobin | mg/mL | 0.025 | 0 | TRUE |
| ICAM-1 | ng/mL | 3.2 | 0 | TRUE |
| IgA | mg/mL | 0.0084 | 0 | FALSE |
| IgE | ng/mL | 14 | 170 | TRUE |
| IGF-1 | ng/mL | 4 | 94 | TRUE |
| IgM | mg/mL | 0.015 | 0 | TRUE |
| IL-16 | pg/mL | 66 | 0 | TRUE |
| IL-18 | pg/mL | 54 | 3 | TRUE |
| IL-1ra | pg/mL | 15 | 17 | TRUE |
| IL-7 | pg/mL | 53 | 209 | TRUE |
| IL-8 | pg/mL | 3.5 | 6 | TRUE |
| Insulin | uIU/mL | 0.86 | 40 | TRUE |
| Leptin | ng/mL | 0.1 | 0 | TRUE |
| Lipoprotein (a) | ug/mL | 3.7 | 0 | TRUE |
| MCP-1 | pg/mL | 52 | 0 | TRUE |
| MDC | pg/mL | 14 | 0 | TRUE |
| MIP-1alpha | pg/mL | 13 | 202 | TRUE |
| MIP-1beta | pg/mL | 38 | 3 | TRUE |
| MMP-3 | ng/mL | 0.2 | 0 | TRUE |
| Myeloperoxidase | ng/mL | 68 | 9 | TRUE |
| Myoglobin | ng/mL | 1.1 | 0 | TRUE |
| PAI-1 | ng/mL | 0.9 | 0 | TRUE |
| Prostate Specific Antigen, | ng/mL | 0.023 | 101 | TRUE |
| Free | | | | |
| Prostatic Acid Phosphatase | ng/mL | 0.034 | 0 | TRUE |
| RANTES | ng/mL | 0.048 | 0 | TRUE |
| Serum Amyloid P | ug/mL | 0.058 | 0 | TRUE |
| SGOT | ug/mL | 3.7 | 80 | TRUE |
| SHBG | nmol/L | 1.3 | 0 | TRUE |
| Stem Cell Factor | pg/mL | 56 | 1 | TRUE |
| Thyroid Stimulating Hormone | uIU/mL | 0.028 | 0 | FALSE |
| Thyroxine Binding Globulin | ug/mL | 0.34 | 0 | TRUE |
| TIMP-1 | ng/mL | 8.4 | 0 | TRUE |
| TNF-alpha | pg/mL | 4 | 233 | TRUE |
| TNF RII | ng/mL | 0.13 | 0 | TRUE |
| VCAM-1 | ng/mL | 2.6 | 0 | TRUE |
| VEGF | pg/mL | 7.5 | 0 | TRUE |
| von Willebrand Factor | ug/mL | 0.4 | 0 | TRUE |

The average pairwise correlation from the sample correlation matrix was also assessed; all samples showed at least an average of 89% correlation to other samples, indicating the biomarker data was consistent across subject samples.

Summary statistics for the biomarkers are shown in Table 3. The distribution of baseline biomarker levels was generally balanced across the three treatment groups.

TABLE 3

| Marker | Mean | SD | Min | Max | ANOVA p¹ |
|---|---|---|---|---|---|
| Adiponectin | 1.330 | 0.762 | −0.713 | 3.585 | 0.525 |
| Alpha.1.Antitrypsin | 1.216 | 0.418 | 0.138 | 2.609 | 0.884 |
| Alpha.2.Macroglobulin | −0.995 | 0.707 | −2.252 | 0.848 | 0.816 |
| Alpha.Fetoprotein | 1.130 | 0.695 | −1.218 | 3.585 | 0.337 |
| Apolipoprotein.A1 | −1.273 | 0.463 | −2.120 | 0.585 | 0.232 |
| Apolipoprotein.CIII | 5.850 | 0.680 | 4.248 | 7.983 | 0.037 |
| Apolipoprotein.H | 7.769 | 0.350 | 6.267 | 9.574 | 0.974 |
| Beta.2.Microglobulin | 0.729 | 0.345 | −0.074 | 1.585 | 0.481 |
| Brain.Derived.Neurotrophic.Factor | 4.406 | 0.539 | 2.036 | 5.322 | 0.626 |
| C.Reactive.Protein | 3.321 | 2.070 | −2.737 | 5.615 | 0.544 |
| Cancer.Antigen.125 | 3.846 | 0.718 | 2.070 | 6.845 | 0.061 |
| Cancer.Antigen.19.9 | 0.747 | 1.579 | −2.000 | 4.170 | 0.731 |
| Carcinoembryonic.Antigen | 0.368 | 0.832 | −0.252 | 3.700 | 0.513 |
| CD40 | −0.904 | 0.540 | −2.644 | 0.379 | 0.533 |
| CD40.Ligand | 2.094 | 1.419 | 0.020 | 6.600 | 0.662 |
| Complement.3 | 0.423 | 0.390 | −0.556 | 1.263 | 0.364 |
| EGF | 6.650 | 1.494 | 2.888 | 9.260 | 0.628 |
| EN.RAGE | 6.236 | 1.153 | 3.459 | 8.071 | 0.564 |
| ENA.78 | 1.100 | 0.808 | −0.474 | 3.907 | 0.814 |
| Eotaxin | 6.580 | 0.690 | 5.358 | 7.966 | 0.372 |
| Factor.VII | 9.260 | 0.628 | 7.539 | 10.834 | 0.706 |
| Ferritin | 6.677 | 1.228 | 3.700 | 9.022 | 0.148 |
| Fibrinogen | −6.238 | 0.392 | −6.673 | −5.059 | 0.239 |
| G.CSF | 2.943 | 0.722 | 2.322 | 4.700 | 0.931 |
| Glutathione.S.Transferase | 1.631 | 0.606 | −0.105 | 2.868 | 0.361 |
| Growth.Hormone | −1.593 | 1.620 | −2.943 | 2.722 | 0.453 |
| Haptoglobin | 1.273 | 0.977 | −1.690 | 3.087 | 0.435 |
| ICAM.1 | 7.053 | 0.445 | 5.492 | 8.459 | 0.152 |
| IgA | 2.485 | 1.218 | 0.290 | 7.300 | 0.606 |
| IgE | 4.923 | 1.612 | 3.807 | 9.430 | 0.863 |
| IGF.1 | 3.606 | 1.403 | 2.000 | 7.055 | 0.509 |
| IgM | −0.022 | 0.716 | −1.737 | 1.926 | 0.513 |
| IL.16 | 9.123 | 0.610 | 7.707 | 10.944 | 0.309 |
| IL.18 | 7.656 | 0.607 | 5.755 | 9.324 | 0.072 |
| IL.1ra | 6.195 | 1.130 | 3.907 | 9.177 | 0.499 |
| IL.7 | 5.937 | 0.432 | 5.728 | 8.028 | 0.860 |
| IL.8 | 4.234 | 1.451 | 1.807 | 9.685 | 0.632 |
| Insulin | 2.403 | 1.830 | −0.218 | 6.870 | 0.405 |
| Leptin | 2.551 | 1.892 | −2.474 | 6.524 | 0.995 |
| Lipoprotein..a. | 5.383 | 1.452 | 3.217 | 9.313 | 0.746 |
| MCP.1 | 7.507 | 0.678 | 5.781 | 9.474 | 0.153 |
| MDC | 8.903 | 0.503 | 7.322 | 10.024 | 0.702 |
| MIP.1alpha | 4.099 | 0.710 | 3.700 | 6.700 | 0.335 |
| MIP.1beta | 7.718 | 0.828 | 5.248 | 10.436 | 0.450 |
| MMP.3 | 3.106 | 1.092 | 0.926 | 7.022 | 0.230 |
| Myeloperoxidase | 9.613 | 1.255 | 6.087 | 11.750 | 0.714 |
| Myoglobin | 3.021 | 0.853 | 1.000 | 5.807 | 0.178 |
| PAI.1 | 7.318 | 0.406 | 5.907 | 8.508 | 0.817 |
| Prostate.Specific.Antigen..Free | −2.824 | 2.051 | −5.442 | 1.000 | 0.593 |
| Prostatic.Acid.Phosphatase | −1.744 | 0.555 | −3.059 | −0.454 | 0.152 |
| RANTES | 4.697 | 0.766 | 2.459 | 6.392 | 0.990 |
| Serum.Amyloid.P | 5.106 | 0.408 | 3.202 | 5.807 | 0.731 |
| SGOT | 2.573 | 0.607 | 1.888 | 4.000 | 0.370 |
| SHBG | 5.044 | 0.751 | 3.459 | 7.313 | 0.598 |
| Stem.Cell.Factor | 7.841 | 0.592 | 6.304 | 9.780 | 0.601 |
| Thyroid.Stimulating.Hormone | 1.462 | 0.741 | 0.380 | 5.000 | 0.810 |
| Thyroxine.Binding.Globulin | 5.939 | 0.341 | 4.322 | 6.794 | 0.950 |
| TIMP.1 | 7.068 | 0.291 | 6.285 | 7.925 | 0.554 |
| TNF.alpha | 2.210 | 0.492 | 2.000 | 5.426 | 0.146 |
| TNF.RII | 1.595 | 0.463 | 0.585 | 2.828 | 0.355 |
| VCAM.1 | 8.498 | 0.319 | 7.864 | 9.468 | 0.558 |
| VEGF | 8.891 | 0.941 | 6.322 | 11.499 | 0.433 |
| von.Willebrand.Factor | 4.820 | 0.646 | 2.787 | 6.150 | 0.845 |

In the golimumab treated groups, multiple markers changed significantly from baseline levels to Week 4 and Week 14. A much more limited set of markers changed in the placebo treated subjects. In general, the differences between the two golimumab dose groups were not significant. The within-subject changes from baseline were compared between golimumab (dosage groups combined) and the placebo group. Approximately half of the markers assayed showed significant differences in change from baseline between golimumab and placebo (Tables 4) and 5) show the markers with significant (p<0.01) differences in change from baseline between the combined golimumab group and the placebo group.

TABLE 4

| | Mean Change From Baseline at Week 4 | | | | |
|---|---|---|---|---|---|
| | | | Golimumab | Golimumab | |
| | Placebo | Placebo | dosed | dosed | Gol vs |
| | Mean | Change p- | Mean | Change p- | Placebo |
| Marker | Change | Value | Change | Value | p-Value |
| Apolipoprotein A1 | −0.072 | 0.248 | 0.141 | 0.000 | 0.003 |
| C-Reactive Protein | −0.265 | 0.246 | −1.875 | 0.000 | 0.000 |
| Complement | −0.016 | 0.798 | −0.258 | 0.000 | 0.001 |
| Component 3 | | | | | |
| Ferritin | −0.045 | 0.547 | −0.314 | 0.000 | 0.005 |
| Haptoglobin | −0.062 | 0.343 | −0.927 | 0.000 | 0.000 |
| ICAM-1 | −0.050 | 0.259 | −0.283 | 0.000 | 0.000 |
| MMP3 | −0.004 | 0.963 | −0.380 | 0.000 | 0.006 |
| Serum.Amyloid.P | −0.056 | 0.088 | −0.326 | 0.000 | 0.000 |
| SHBG | −0.047 | 0.392 | 0.132 | 0.001 | 0.010 |
| TNFRII | −0.029 | 0.409 | −0.172 | 0.000 | 0.002 |

TABLE 5

| | Mean Change From Baseline at Week 14 | | | | |
|---|---|---|---|---|---|
| | | | Golimumab | Golimumab | |
| | Placebo | Placebo | dosed | dosed | Gol vs |
| | Mean | Change p- | Mean | Change p- | Placebo |
| Marker | Change | Value | Change | Value | p-Value |
| C.Reactive.Protein | 0.027 | 0.905 | −2.240 | 0.000 | 0.000 |
| Complement.3 | 0.052 | 0.523 | −0.305 | 0.000 | 0.000 |
| ENA-78 | 0.068 | 0.254 | −0.205 | 0.000 | 0.000 |
| Ferritin | −0.123 | 0.099 | −0.443 | 0.000 | 0.002 |
| Haptoglobin | 0.166 | 0.073 | −1.020 | 0.000 | 0.000 |
| ICAM-1 | −0.032 | 0.517 | −0.334 | 0.000 | 0.000 |
| MIP-1beta | −0.122 | 0.219 | −0.794 | 0.000 | 0.000 |
| MMP3 | 0.184 | 0.047 | −0.531 | 0.000 | 0.000 |
| PAI-1 | 0.039 | 0.394 | −0.249 | 0.000 | 0.000 |
| RANTES | 0.210 | 0.029 | −0.182 | 0.022 | 0.002 |
| Serum Amyloid.P | −0.006 | 0.911 | −0.388 | 0.000 | 0.000 |
| SHBG | −0.102 | 0.164 | 0.193 | 0.002 | 0.002 |
| Thyroxine.Binding | −0.006 | 0.868 | −0.124 | 0.000 | 0.010 |
| Globulin | | | | | |
| TIMP-1 | 0.052 | 0.128 | −0.140 | 0.000 | 0.000 |
| TNFalpha | 0.018 | 0.631 | −0.106 | 0.000 | 0.010 |
| TNFRII | 0.008 | 0.828 | −0.198 | 0.000 | 0.000 |
| VEGF | 0.044 | 0.482 | −0.506 | 0.000 | 0.000 |

### EXAMPLE 2

### Marker and Association

In order build a predictive model or algorithm, the marker data was evaluated in association with the study clinical endpoints. There were six clinical endpoints in this study, defined as ASAS20 Week 14, ASAS20 Week 24, Change in BASMI Week 14, Change in BASFI Week 14, and the Change in BASDAI Week 14. These study endpoints are generally accepted clinical methods to evaluate disease status in patients. The 100 patients in the protein biomarker sub-study and the study endpoints collected are shown below (Table 6).

TABLE 6

| | | | | | Patients | Clinical |
|---|---|---|---|---|---|---|
| | | | | | who | Endpoint |
| | Enrolled in | Baseline | Week 4 | Week 14 | qualified | Data |
| | Protein | patient | patient | patient | for Early | Available |
| Treatment | Biomarker | Data | Data | Data | Escape at | at Weeks |
| Group | sub- study | Collected | Collected | Collected | Week 16 | 14/24 |
| Placebo | 24 | 24/24 | 24/24 | 24/24 | 14/24 | 24/24 |
| | | (100%) | (100%) | (100%) | (58%) | (100%) |
| Gol 50 mg | 37 | 37/37 | 37/37 | 37/37 | 9/37 | 37/37 |
| | | (100%) | (100%) | (100%) | (24%) | (100%) |
| Gol 100 mg | 39 | 39/39 | 39/39 | 39/39 | 9/39 | 39/39 |
| | | (100%) | (100%) | (100%) | (23%) | (100%) |
| Total | 100 | 100/100 | 100/100 | 100/100 | 32/100 | 100/100 |
| | | (100%) | (100%) | (100%) | (32%) | (100%) |

The clinical response primary endpoints are shown in Table 7 where the entries represent responder/total for that group. While not the main focus of the biomarker substudy, it is still helpful to the interpretation of the study to assess the treatment effect on clinical endpoints within this cohort. As shown in Table 7, the response of the golimumab treatment groups were significantly superior to placebo across the range of clinical endpoints assessed, with the exception of BASMI.

TABLE 7

| Endpoint | Gol 100 mg | Gol 50 mg | Placebo | Overall | Gol vs Placebo p |
|---|---|---|---|---|---|
| ASAS20 | 26/39 (67%) | 24/37 (65%) | 4/24 (17%) | 54/100 (54%) | <0.0001 |
| Wk 14 | | | | | |
| ASAS20 | 24/39 (62%) | 23/37 (62%) | 4/24 (17%) | 51/100 (51%) | <0.0001 |
| Wk 24 | | | | | |
| Early | 9/39 (23%) | 9/37 (24%) | 14/24 (58%) | 32/100 (32%) | 0.002 |
| Escape | | | | | |
| ΔBASMI | 23/39 (59%) | 24/37 (65%) | 17/24 (71%) | 64/100 (64%) | 0.474 |
| Wk 14 | | | | | |
| ΔBSF | 23/39 (59%) | 23/37 (62%) | 4/24 (17%) | 50/100 (50%) | 0.0003 |
| Wk 14 | | | | | |
| ΔBASDAI | 25/39 (64%) | 21/37 (57%) | 4/24 (17%) | 50/100 (50%) | .0003 |
| Wk 14 | | | | | |

Within the study patients participating the protein marker study, there was a significant association of sex with three of the six clinical endpoints (Table 8). Sex was also significantly associated with many of the protein biomarkers. For this reason, sex was used as a covariate to adjust the models that tested for associations between biomarkers values and clinical endpoints. Without this adjustment, markers that are correlated with sex (e.g. Prostate Specific Antigen) would appear to be associated with clinical endpoints, but that association would be an artifact of the sex/endpoint association. CRP is a marker commonly associated with AS, however, in this study the baseline values of CRP were not statistically correlated with the clinical endpoints.

TABLE 8

| Endpoint | Sex | Age | Weight | CRP |
|---|---|---|---|---|
| ASAS20 Wk 14 | 0.012 | 0.489 | 0.134 | 0.226 |
| ASAS20 Wk 24 | 0.036 | 0.936 | 0.323 | 0.186 |
| Early Escape | 0.417 | 0.830 | 0.714 | 0.628 |
| ΔBASMI Wk 14 | 0.381 | 0.681 | 0.155 | 0.114 |
| ΔBSF Wk 14 | 0.004 | 0.608 | 0.009 | 0.455 |
| ΔBASDAI Wk 14 | 0.264 | 0.235 | 0.634 | 0.363 |

### EXAMPLE 3

### Prediction Model Building

Biomarkers were assessed for association at baseline, week 4, and week 14. Several findings emerged from these analyses. Few of the 92 markers examined were significantly associated with clinical response. Markers that did showed significant effects, and the marker and endpoint relationship for these markers, was generally consistent across the several primary and secondary endpoints. As there was no dose effect on the clinical outcomes, the data used was combined golimumab treatment groups (all patients receiving golimumab). Biomarkers were assessed for an association at baseline, week 4, and week 14.

All analysis was performed using R (R: A Language and Environment for Statistical Computing, 2008, Author: R Development Core Team, R Foundation for Statistical Computing, Vienna, Austria, ISBN 3-900051-07-0). Change from baseline was tested using one-sample t-tests. Association of clinical factors with baseline biomarkers was evaluated using robust linear regression models. Robust logistic regression models were used to test for the association of biomarkers with clinical endpoints. Clinical endpoint variables that were Yes/No used a 1/0 coding. Clinical endpoints that were continuous were converted into 1/0 variables for this analysis by applying a threshold at the median value of all subjects.

The baseline markers identified consistently across timepoints and clinical endpoints were leptin, haptoglobin, insulin, ENA78, and apoliproprotein C3, osteocalcin, P1NP, and IL6 (by EIA). Each of these markers was significant in at least three clinical endpoints, and had an odds ratio of greater than 1.5 for at least one endpoint. For these markers, Table 9 shows the odds ratios and p-values for their association with clinical endpoints. In Table 9, the odds ratio (OR) represents the increased odds of clinical response for a 1 unit change on the log 2 scale, or a doubling on the linear scale.

To increase the reliability of the results this study, the focus was on identifying markers that showed significant association at multiple timepoints across multiple endpoints. At baseline, the multiplex-determined markers identified consistently across clinical endpoints were leptin, haptoglobin, insulin, ENA78, and apoliproprotein C3. In addition, single ELISA testing of the serum samples identified osteocalcin, P1NP, and IL-6. Each of these eight markers was had a p-value of less than 0.05 in at least three clinical endpoints, and had an odds ratio (OR) of greater than 1.5 for at least one endpoint. For these markers, Table 9 shows the odds ratios and p-values for their association with clinical endpoints. The OR represents the increased odds of a clinical response for a 1 unit change on the log 2 scale, or a doubling on the linear scale.

TABLE 9

| | | | | | Change in | | Change in | |
|---|---|---|---|---|---|---|---|---|
| | ASAS20 | | ASAS20 | | BASFI | | BASDAI | |
| | Wk 14 | | Wk 24 | | at Wk 14 | | at Wk 14 | |
| Marker | OR | p | OR | p | OR | p | OR | p |
| Leptin | 0.64 | 0.041 | 0.063 | 0.029 | 0.62 | 0.027 | 0.79 | 0.207 |
| Haptoglobin | 1.70 | 0.046 | 1.25 | 0.351 | 1.72 | 0.040 | 1.70 | 0.034 |
| Insulin | 0.63 | 0.009 | 0.71 | 0.030 | 0.66 | 0.013 | 0.77 | 0.076 |
| ApoC3 | 0.35 | 0.019 | 0.60 | 0.195 | 0.41 | 0.036 | 0.69 | 0.335 |
| ENA78 | 2.00 | 0.080 | 2.31 | 0.036 | 2.44 | 0.031 | 3.12 | 0.0098 |
| Osteocalcin | 10.88 | 0.001 | 1.97 | 0.130 | 10.14 | 0.002 | 3.13 | 0.033 |
| P1NP | 5.94 | 0.004 | 2.54 | 0.049 | 4.20 | 0.011 | 2.47 | 0.049 |
| IL6 | 1.80 | 0.017 | 1.90 | 0.009 | 1.47 | 0.081 | 1.72 | 0.014 |

The markers where early (week 4) change from baseline was predictive consistently across timepoints and clinical endpoints were Haptoglobin, Serum Amyloid, CRP, Alpha-1 Antitrypsin, vonWillebrand Factor, Complement Factor 3, and the serum marker IL-6 (ELISA). Each of these seven markers was significant in at least 3 clinical endpoints, and had an odds ratio of greater than 3 for at least one endpoint. For these markers, Table 10 shows the odds ratios and p-values for their association with clinical endpoints.

TABLE 10

| | | | | | Change in | | Change in | |
|---|---|---|---|---|---|---|---|---|
| | ASAS20 | | ASAS20 | | BASFI | | BASDAI | |
| | Wk 14 | | Wk 24 | | at Wk 14 | | at Wk 14 | |
| Marker | OR | p | OR | p | OR | p | OR | p |
| Haptoglobin | 0.20 | .007 | 0.31 | .014 | 0.23 | .006 | 0.17 | .002 |
| Serum Amyl. P | 0.30 | .095 | 0.16 | .021 | 0.13 | .013 | 0.21 | .036 |
| CRP | 0.72 | .025 | 0.70 | .013 | 0.69 | .010 | 0.74 | .025 |
| A1 Anti-trypsin | 0.04 | .018 | 0.06 | .018 | 0.09 | .039 | 0.09 | .032 |
| vonWil Factor | 0.54 | .127 | 0.14 | .005 | 0.28 | .019 | 0.73 | .392 |
| Complement3 | 0.02 | .004 | 0.03 | .004 | 0.02 | .003 | 0.01 | .001 |
| IL6 (ELISA) | 0.36 | .003 | 0.42 | .004 | 0.52 | .013 | 0.43 | .002 |

### Placebo

In contrast to the biomarker/clinical endpoint associations observed within the golimumab treated group, there was little if any association of biomarker values to clinical endpoint responses within the placebo groups (not shown). This result serves as an internal control or benchmark for the more significant biomarker results seen in the golimumab biomarker analyses.

### Baseline Biomarker Prediction Methods

Classification and Regression Tree (CART) predictive models were developed that were used to determine which biomarkers could be used to predict the long term clinical response of patients to treatment. All prediction models employed Leave one out cross validation. The CART models are displayed in the form of a decision tree (FIG. 1-6). The nodes of the tree are labeled with a class prediction (Yes for a predicted clinical endpoint responder, No for a predicted clinical non-responder) and two numbers (x/y, where x is the actual number of non responders in the study who would fall into that node and y is the actual number of responders in the study who would fall into that node). The overall accuracy of the model is the number of x's across the ‘No’ end nodes plus the number of y′s across the “Yes’ end nodes. Models were developed for the primary clinical endpoint, ACR20 at Week 14, as well as for selected secondary clinical endpoints. In general, the secondary endpoint models were very similar to the primary endpoint models in terms of their sensitivity and specificity.

The predictive models were used to determine which biomarkers could be used to predict the response of the patients to treatment. One model was developed based on values obtained at baseline for markers analyzed by the multiplex assay and using the ASAS20 (primary) endpoint (FIG. 1). The analysis of the sample results using the model showed that when the model was applied to the samples, the model was correct in 61/76 (80%) of the patients tested. This means that in the patients samples analyzed with the model, in 80% of the patients the results could predict their clinical response (ASAS20) at Week 14. A diagram of the model is given in FIG. 1. The biomarker model uses leptin as the initial classifier: that is, patients with leptin above or equal to 3.8 (log scale) are predicted to be nonresponders. Those patients with leptin levels below 3.8 are then classified based on the use of a secondary marker, CD40 ligand. The patients with a CD40 ligand result above 1.05 are predicted to be responders, while patients with leptin levels below 3.8 and CD40 ligand below 1.05 predicted to be non-responders. The sensitivity of the prediction using the model was 86%. The specificity of the results using the model was 88%.

A prediction model for the BASDAI endpoint is shown in FIG. 2. Different biomarkers were selected for this model and the overall accuracy of the BASDAI model is similar to the ASAS20 model. The algorithm in FIG. 2 is based on TIMP-1 level greater than or equal to 7.033 (log scale) as the initial classifier of response to anti-TNF therapy. Patients with TIMP-1 level greater or equal to 7.033 are further classified using G-CSF less than 3.953 as a predicted responder and G-CSF greater than or equal to 3.953 as a predicted non-responder. Patients with TIMP-1 level less than 7.033 are further classified using PAP levels where a level of less than −1.287 is predictive of a responder and patients with a level greater than −1.287 are further classified based on MCP-1 levels, where MCP-1 less than 7.417 is predictive of a responder and MCP-1 greater than or equal to 7.417 is predictive of a nonresponder.

When the markers analyzed using individual EIA assays (non-multiplexed assays) and a 3 plex assay (Luminex) were included in the CART analysis, the algorithms (decision trees) resulting relied on osteocalcin as the initial classifier whether the clinical endpoint was ASAS20 or BASDAI (FIGS. 3 and 4, respectively). It was found that the additional markers enhanced the predictive ability of the panel of markers. The accuracy of the baseline biomarker/serum biomarker model was 67/76 (88%) for prediction of clinical response as assessed by ASAS20 at Week 14 (FIG. 3). This biomarker model uses osteocalcin (assayed by individual EIA) as the initial classifier: patients with osteocalcin greater than or equal to 3.878 (log scale) are predicted to be responders; patients with osteocalcin below 3.878 are classified based on PAP. The model accuracy was 88%, sensitivity was 90%, and model specificity was 84%.

In a similar analysis, a prediction model for the BASDAI endpoint is shown in FIG. 4. In this case, the BASDAI and ASAS20 models turned out to be very similar (both included osteocalcin and PAP) the BASDAI model added insulin as one additional classifier). Model accuracy was 61/76 (80%) for prediction of BASDAI clinical response.

Baseline Concentration and Change from Baseline at Week 4

An additional prediction model using the multiplex data was developed to determine if the change in a biomarker at Week 4 of treatment could be included in predicting the clinical outcome at Week 14. An algorithm for predicting ASAS20 is displayed in FIG. 5. As with the baseline only algorithm for predicting ASAS20, the baseline leptin is the initial classifier: patients with leptin greater than or equal to 3.8 (log scale) are predicted to be non-responders; patients with leptin below 3.8 are further classified based on two additional predictors: i) change in complement 3, and ii) baseline VEGF. In this model, the accuracy was 64/76 (84%) for predicting clinical response (ASAS20) at Week 14. The sensitivity of the model was 92%, and the specificity was 81%.

A prediction model for the BASDAI endpoint is shown in FIG. 6. While the overall accuracy of the BASDAI model is similar to the ASAS20 model, different biomarkers were selected and used in this analysis: the initial marker was change in Complement component 3 from Week 0 to Week 4 where patients with a decrease of less than 0.233 (log scale) are predicted to be responders; patients with a greater or equal to 0.2333 decrease in Complement component 3 are further classified based on baseline ferritin, where if the ferritin value is greater than the cutoff value of 7.774 the patient is classified as a predicted responder and where ferritin is less than 7.774 the patient is classified as a predicted nonresponder; the subset of those predicted as a nonresponder based on ferritin are further classified based on the change in ICAM-1 levels where those with a decrease in ICAM-1 between Week 0 and Week 4 of greater than or equal to 0.02204 are classified as a predicted responders and the remaining patients with a decrease in ICAM-1 between Week 0 and Week 4 of less than 0.02204 classified as predicted nonresponders.

### Summary

The results of the protein biomarker study showed that multiple biomarkers changed significantly as a consequence of golimumab therapy. In contrast, few biomarker changes were observed in the placebo control arm. Two types of novel biomarker-based clinical response prediction models were developed, one that used baseline biomarker values only to predict a patients clinical response, another that used early (Week 4) changes in biomarker values to predict longer term (Weeks 14, 24) clinical responses. The models suggest that a subset of the markers have changes associated with clinical response to golimumab, as opposed to simply being non-specific effects of treatment. This can be concluded from the robust logistical regression analyses looking across multiple clinical endpoints

Importantly, the marker values (either at baseline or the week 4 changes) preceded the clinical outcomes. This shows that a panel of biomarkers can be developed that can be used to predict with good accuracy the eventual response or non-response of AS patients to golimumab treatment.

The best biomarker model (based on specificity and sensitivity) of clinical response (signs and symptoms) to golimumab included baseline levels of osteocalcin and prostatic acid phosphatase as shown in FIGS. 3 and 4.

What is to be claimed:

1. A method for predicting the response of a patient having the diagnosis of ankylosing spondylitis to anti-TNFalpha therapy, the method comprising:
a) determining the concentration of at least one serum marker selected from the group consisting of leptin, CD40 ligand, TIMP-1, prostatic acid phosphatase (PAP), G-CSF, MCP-1, complement component 3, VEGF, osteocalcin, ferritin, and ICAM-1; and
b) comparing the concentration determined to a cutoff value determined by analyzing a set of values of serum concentrations of the marker from patients diagnosed with AS who received anti-TNFa therapy and were classified as a responder or a non-responder based on one or more clinical endpoints.

2. The method of claim 1, wherein an additional marker concentration is determined in the serum selected from the group consisting of haptoglobin, serum amyloid, CRP, alpha-1 antitrypsin, von Willebrand Factor, and insulin in a blood or serum sample from said patient.

3. A method for predicting the response of a patient having the diagnosis of ankylosing spondylitis to anti-TNFalpha therapy, the method comprising:
a) determining the concentration of leptin and CD40 ligand in a blood or serum sample from said patient; and
b) comparing said concentration of leptin in the AS sample to a leptin cutoff value whereby if the concentration is determined to be greater than or equal to the cutoff value, the patient is predicted to be a non-responder to anti-TNFalpha therapy, and, if the serum value of leptin is below the cutoff value,
c) comparing the concentration of CD40 ligand in the patient's sample to a CD40 ligand cutoff value, wherein a concentration of CD40 above or equal to the CD40 ligand cutoff value is indicative of the patient's response to TNFalpha therapeutic, and a value below the CD40 ligand value and leptin below the leptin cutoff value is predictive of a non-responder to TNFalpha neutralizing therapeutic.

4. The method of claim 3, wherein the sample is serum.

5. The method of claim 4 where the concentration of leptin in serum is log transformed and the leptin cutoff value is 3.804.

6. The method of claim 3, wherein concentration of CD40 in serum is log transformed and the CD40 cutoff value is 1.05.

7. The method of claim 3, wherein the determining step is performed simultaneously.

8. A method of claim 3, wherein the determining step is performed by a computer-assisted device.

9. The method of any of claims 1-5 wherein said patient has been treated with a non-TNF neutralizing therapeutic.

10. A method for predicting the response of a patient having the diagnosis of ankylosing spondylitis to anti-TNFalpha therapy, the method comprising:
a) determining the concentration of osteocalcin, prostatic acid phosphatase, and insulin in a blood or serum sample from said patient; and
b) comparing said concentration of osteocalcin in the AS sample to osteocalcin cutoff value whereby if the concentration is determined to be greater than or equal to the cutoff value, the patient is predicted to be a non-responder to anti-TNFalpha therapy, and a if the serum value of osteocalcin is below the cutoff value,
c) comparing the concentration of prostatic acid phosphatase in the patient's sample to a prostatic acid phosphatase cutoff value, wherein a concentration of prostatic acid phosphatase above or equal to the prostatic acid phosphatase cutoff value the patient is predicted to be a responder to TNFalpha therapeutic, and a value below the prostatic acid phosphatase cutoff value, and, optionally,
d) classifying the patient as a predicted to be a non-responder based the clinical outcome assessed by ASAS20 or further classifying the patient by comparing the concentration in the patients serum of insulin to an insulin cutoff value wherein an insulin value below the insulin cutoff value classifies the patient as predicted to be a responder and an insulin value greater than or equal to a cutoff value classifies the patient as predicted to be a nonresponder to TNFalpha neutralizing therapeutic as assessed by BASDAI.

11. The method of claim 10, wherein the sample is serum.

12. The method of claim 11 where the concentration of osteocalin in serum is log transformed and the osteocalcin cutoff value is 3.9

13. The method of claim 10, wherein concentration of prostatic acid phosphatase in serum is log transformed and the prostatic acid phosphatase cutoff value is 1.4.

14. The method of claim 10, wherein concentration of insulin in serum is log transformed and the insulin cutoff value is 2.711.

15. The method of claim 10, wherein the determining step is performed simultaneously.

16. A method of claim 15, wherein the determining step is performed by a computer-assisted device.

17. A method for predicting the response of a patient having the diagnosis of ankylosing spondylitis to anti-TNFalpha therapy, the method comprising:
a) determining the concentration of osteocalcin and prostatic acid phosphatase in a blood or serum sample from the patient; and
b) comparing said concentration of osteocalcin in the AS sample to an osteocalcin cutoff value whereby if the concentration is determined to be greater than or equal to the cutoff value, the patient is predicted to be a non-responder to anti-TNFalpha therapy, and a if the serum value of osteocalcin is below the cutoff value,
c) comparing the concentration of prostatic acid phosphatase in the patient's sample to a prostatic acid phosphatase cutoff value, wherein a concentration of prostatic acid phosphatase above or equal to the prostatic acid phosphatase cutoff value the patient is predicted to be a responder to TNFalpha therapeutic, and a value below the prostatic acid phosphatase cutoff value,
d) classifying the patient as a predicted to be a non-responder based the clinical outcome assessed by.

18. A method for predicting the response of a patient having the diagnosis of ankylosing spondylitis to anti-TNFalpha therapy, the method comprising:
a) determining the concentration of TIMP-1 and prostatic acid phosphatase, GCSF, and MCP-1 in a blood or serum sample from said patient; and
b) comparing said concentration of TIMP-1 in the AS sample to a TIMP-1 cutoff value whereby if the concentration is determined to be greater than or equal to the TIMP-1 cutoff value, the patient will be further classified, and a if the serum value of TIMP-1 is below the cutoff value,
c) comparing the concentration of prostatic acid phosphatase in the patient's sample to a prostatic acid phosphatase cutoff value, wherein a concentration of prostatic acid phosphatase below the prostatic acid phosphatase cutoff value the patient is predicted to be a responder to TNFalpha therapeutic, and a value greater than or equal to the prostatic acid phosphatase cutoff value requires that the patient be further classified,
d) comparing the concentration in the patients serum of MCP-1 to a MCP-1 cutoff value wherein a MCP-1 value below the MCP-1 cutoff value classifies the patient as predicted to be a responder and a MCP-1 value greater than or equal to a cutoff value classifies the patient as predicted to be a nonresponder to TNFalpha neutralizing therapeutic as assessed by BASDAI.

19. The method of claim 18 wherein, when the patient's serum has a level of TIMP-1 greater than or equal to the TIMP-1 cutoff value, the level of G-CSF in the patient's serum is compared to a G-CSF cutoff value where in if the G-CSF level in the patients serum is below a G-CSF cutoff value the patient is classified as predicted to respond to anti-TNF therapy as assessed by BASDAI and if the G-CSF value is greater than or equal to the G-CSF cutoff value the patient is classified as predicted to be a nonresponder to anti-TNF therapy as assessed by BASDAI.

20. The method of claims 18 and 19 wherein the TIMP-1 cutoff value is 7.03.

21. A method for predicting the response of a patient having the diagnosis of ankylosing spondylitis to anti-TNFalpha therapy, the method comprising:
a) determining the change in complement component 3 (C3) concentration from a baseline sample and a Week 4 sample and ferritin at baseline and the change in marker concentration from baseline and Week 4 ICAM-1 in a blood or serum sample from said patient; and
b) comparing the change in said concentration of C3 in the AS patient's serum sample taken prior to the initiation of anti-TNF therapy to the concentration of C3 in the AS patient's serum sample taken at Week 4 after initiation of anti-TNF therapy to a C3 cutoff value whereby if the change in concentration is determined to be less than the C3 cutoff value, the patient is classified as predicted to respond to anti-TNF therapy, classifying a patient with a change in serum concentration of C3 greater than or equal to the C3 cutoff value using the baseline value of ferritin in the patient's sample compared to a ferritin cutoff value wherein a value greater than or equal to the cutoff value caused the patient to be predicted to be a responder to anti-TNFalpha therapy, and a if the serum value of ferritin level is below the cutoff value,
c) comparing the change in said concentration of ICAM-1 in the AS patient's serum sample taken prior to the initiation of anti-TNF therapy to the concentration of ICAM-1 in the AS patient's serum sample taken at Week 4 after initiation of anti-TNF therapy to a ICAM-1 cutoff value whereby if the change in ICAM-1 concentration is determined to be greater than or equal to the ICAM-1 cutoff value, the patient is classified as predicted to respond to anti-TNF therapy and if the the change in ICAM-1 concentration is determined to be less than the ICAM-1 cutoff value the patient is classified as a predicted nonresponder.

22. The method of claims 21 wherein the C3 change cutoff value is −0.233.

23. A computer based system for applying a prediction algorithm to a set of data obtained from a patient diagnosed with ankylosing spondylitis to be treated with an anti-TNFalpha therapeutic and assessed using one or more clinical endpoints after treatment, comprising:
a computation station for receiving and processing a patient data set in computer readable format, said computation station comprising a trained neural network for processing said patient data set and producing an output classification, wherein said trained neural network is trained with a method for preprocessing a patient data set, comprising:
a) selecting patient biomarkers associated with AS,
b) statistically and/or computationally testing discriminating power of the selected patient biomarkers individually in linear and/or non-linear combination for indicating the response or non-response of a patient based on a clinical endpoint,
c) applying statistical methods for the derivation of secondary inputs to the neural network that are linear or non-linear combinations of the original or transformed biomarkers,
d) selecting only those patient biomarkers or derived secondary inputs that show discriminating power; and
e) training the computer-based neural network using the preprocessed patient biomarkers or derived secondary inputs.

24. The computer based system of claim 23, wherein the output classification is whether the patient will respond or not respond to anti-TNFa therapy and the clinical endpoints are ASA20 or BASDAI and the biomarkers are patient sex, leptin, CD40 ligand, TIMP-1, MCP-1, G-CSF, PAP, osteocalcin, insulin, VEGF, ferritin, complement component 3, ICAM-1 or any combination of the biomarkers.

25. A device for predicting whether a patient diagnosed with ankylosing spondylitis to be treated with an anti-TNFalpha therapeutic will respond or not respond to therapy as assessed by the one or more clinical endpoints, comprising
a) a test strip comprising an antibody specific for a marker associated with AS patient response or non-response to anti-TNFa therapy selected from the group consisting of leptin, CD40 ligand, TIMP-1, MCP-1, G-CSF, PAP, osteocalcin, insulin, VEGF, ferritin, complement component 3, or ICAM-1, and a second antibody labeled with a detectable label;
b) detecting the signal produced by the label using a reader capable of processing the signal; and
c) processing the data obtained from the processing of the signal into a result indicative of a predetermined concentration of the marker in the sample.

26. The device of claim 25, wherein the reader is a human.

27. The device of claim 25, wherein the reader is a reflectometer.

28. A prognostic test kit for use in predicting whether a patient diagnosed with ankylosing spondylitis to be treated with an anti-TNFalpha therapeutic will respond or not respond to therapy as assessed by the one or more clinical endpoints, comprising: a preprepared substrate capable of quantitating the presence of one or more markers in a patient sample selected from the group consisting of leptin, CD40 ligand, TIMP-1, MCP-1, G-CSF, PAP, osteocalcin, insulin, VEGF, ferritin, complement component 3, ICAM-1 or any combination thereof.
